(19) United States
(12) Patent Application Publication
(10) Pub. No.: US 2005/0163850 A1
(43) Pub. Date: Jul. 28, 2005

(54) Administration of levodopa and carbidopa

(76) Inventors: Patrick S.L. Wong, Burlingame, CA (US); Dong Yan, Cupertino, CA (US); Stephen Hwang, Palo Alto, CA (US); George Guittard, Cupertino, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/978,252

(22) Filed: Oct. 29, 2004

Related U.S. Application Data
(60) Provisional application No. 60/519,509, filed on Nov. 12, 2003.
(60) Provisional application No. 60/516,259, filed on Oct. 31, 2003.

(51) Int. Cl. A61K 9/24; A61K 31/205

(52) U.S. Cl. 424/473; 514/554

(57) ABSTRACT

Disclosed are substances, compositions, dosage forms and methods that comprise levodopa and/or carbidopa.

### CROSS-REFERENCE TO RELATED APPLICATIONS

This application claims the benefit of U.S. provisional patent application No. 60/519,509, filed Nov. 12, 2003, and of U.S. Provisional Application number 60/516,259, filed Oct. 31, 2003, both applications are incorporated herein by reference in their entirety.

### FIELD OF THE INVENTION

The invention relates to substances, compositions, dosage forms and methods that comprise levodopa and/or carbidopa.

### BACKGROUND

Parkinson's disease is a progressive, neurodegenerative disorder of the extrapyramidal nervous system affecting the mobility and control of the skeletal muscular system. Its characteristic features include resting tremor, rigidity, and bradykinetic movements.

The gold standard of present therapy for Parkinson's Disease is the drug levodopa,(also called L-dopa). Levodopa, an aromatic amino acid, is a white, crystalline compound, slightly soluble in water, with a molecular weight of 197.2. It is designated chemically as (−)-L-(alpha)-amino-(beta)-(3,4-dihydroxybenzene) propanoic acid. Its empirical formula is C₉H₁₁NO₄, and its structural formula is:

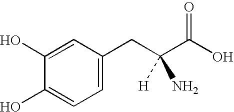

Current evidence indicates that symptoms of Parkinson's disease are related to depletion of dopamine in the corpus striatum. Administration of dopamine is ineffective in the treatment of Parkinson's disease apparently because it does not cross the blood-brain barrier. Levodopa, however, can cross the blood-brain barrier by way of the large neutral amino acid carrier transport system. Presumably levodopa is converted to dopamine in the brain. This is thought to be the mechanism whereby levodopa relieves symptoms of Parkinson's disease.

Usually levodopa is given combined with carbidopa. Carbidopa, an inhibitor of aromatic amino acid decarboxylation, is a white, crystalline compound, slightly soluble in water, with a molecular weight of 244.3. It is designated chemically as (−)-L-((alpha)-hydrazino-((alpha)-methyl-(beta)-(3,4-dihydroxybenzene) propanoic acid monohydrate. Its empirical formula is C₁₀H₁₄N₂O₄.H₂ O, and its structural formula is

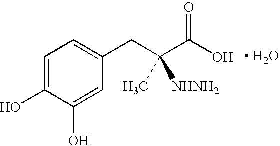

When levodopa is administered orally it is rapidly decarboxylated to dopamine in extracerebral tissues so that only a small portion of a given dose is transported unchanged to the central nervous system. Carbidopa inhibits the decarboxylation of peripheral levodopa, making more levodopa available for transport to the brain. When coadministered with levodopa, carbidopa increases plasma levels of levodopa and reduces the amount of levodopa required to produce a given response by about 75%. Carbidopa prolongs the plasma half-life of levodopa from 50 minutes to 1.5 hours and decreases plasma and urinary dopamine and its major metabolite, homovanillic acid.

The above compounds have been incorporated into a variety of immediate release oral dosage forms, such as Sinemet™ (carbidopa and levodopa). Conventional controlled release versions of some of these oral dosage forms have also been developed, such as Sinemet™ CR.

A problem with these conventional oral dosage forms is that they do not provide for particularly good control of Parkinson's disease as compared with other therapies. For instance, long-term studies of intraduodenal infusion of levodopa/carbidopa solutions found that motor fluctuations in parkinsonian patients can be markedly reduced. These infusion techniques show a positive effect even after 4-7 years of continuous duodenal infusion. D. Nilsson et al., “Duodenal levodopa infusion in Parkinson's disease—long term experience”, Acta Neurol Scan 104:343-348 (2001).

Such intraduodenal infusion has even been shown to be superior to oral dosing of levodopa & carbidopa using conventional oral controlled release dosage forms. D. Nyholm et al., “Optimizing Levodopa Pharmacokinetics: Intestinal Infusion Versus Oral Sustained-Release Tablets”, Clin. Neuropharmacology 26(3):156-163, (2003). The average intraindividual coefficient of variation for the plasma levodopa concentrations after oral therapy was 34% and was significantly lower (14%, p<0.01) during continuous infusion. Hourly video evaluations showed a significant increase in ON time (evidenced by a normal or near normal ability to perform specified motor tasks) during infusion and a significant decrease in OFF time (severe parkinsonism) and dyskinesia. Peak-effect dyskinesias are abolished, resulting from elimination of peaks of levodopa and central dopamine concentrations.

Despite the superior therapeutic performance of such long-term infusion strategies, they remain difficult and inconvenient to implement, as they require that the patient be tethered to a pump for their entire waking hours. Additionally, pump malfunctions can occur, resulting in severe problems for the Parkinson's patient.

Accordingly, substances, compositions, dosage forms, and methods are needed to address the problems noted above.

### SUMMARY OF THE INVENTION

In an aspect, the invention relates to a substance comprising: a complex comprising levodopa and a transport moiety.

In an aspect, the invention relates to a method comprising: providing an alkyl sulfate salt; converting the alkyl sulfate salt to an acid form of the alkyl sulfate; contacting levodopa with the acid form of the alkyl sulfate to form a levodopa-alkyl sulfate complex; and isolating the complex.

In an aspect, the invention relates to a substance comprising: a complex comprising carbidopa and a transport moiety.

In an aspect, the invention relates to a method comprising: providing an alkyl sulfate salt; converting the alkyl sulfate salt to an acid form of the alkyl sulfate; contacting carbidopa with the acid form of the alkyl sulfate to form a levodopa-alkyl sulfate complex; and isolating the complex.

In an aspect, the invention relates to an oral dosage form comprising: (i) an oral controlled delivery dosing structure comprising structure that controllably delivers a substance that comprises levodopa and a substance that comprises carbidopa; wherein at least a portion of the substance that comprises levodopa and a portion substance that comprises carbidopa are contained by the controlled delivery dosing structure; and
wherein the controlled delivery dosing structure is adapted to controllably deliver the portion of the substance that comprises levodopa and the portion of the substance that comprises carbidopa contained by the controlled delivery dosing structure at rates that are effective to, after a single administration of the dosage form to a patient:
a. provide a levodopa Cmax ranging from about 236 to about 988 ng/mL,
b. provide a levodopa AUC from about 3676 to about 15808 h·ng/mL, and
c. maintain a levodopa plasma drug concentration that is at least about fifteen percent of the Cmax throughout a window of at least about ten hours duration.
d. provide a carbidopa Cmax ranging from about 1 to about 500 ng/ml μmol/L,
e. provide an carbidopa AUC from about 20000 to about 200000 h·ng/mL, and
f. maintain a carbidopa plasma drug concentration that is at least about fifteen percent of the Cmax throughout a window of at least about ten hours duration.

In an aspect, the invention relates to an oral controlled delivery dosage form comprising an oral controlled delivery dosing structure comprising structure that controllably delivers a substance that comprises levodopa; wherein at least a portion of the substance that comprises levodopa is contained by the controlled delivery dosing structure; and wherein the controlled delivery dosing structure is adapted to controllably deliver the portion of the substance that comprises levodopa contained by the controlled delivery dosing structure at an ascending rate of release effective to, after a single administration of the dosage form to a patient, provide a substantially zero order levodopa plasma profile for a window of at least about six hours duration

In an aspect, the invention relates to a composition comprising: levodopa; an alkyl sulfate salt; and a pharmaceutically-acceptable carrier.

### BRIEF DESCRIPTION OF THE FIGURES

The following figures are not drawn to scale, and are set forth to illustrate various embodiments of the invention.

FIG. 1 is a diagram of epithelial cells of the gastrointestinal tract, illustrating two transport routes of drugs through the epithelium of the G.I. tract.

FIG. 2 shows a diagram of a liquid osmotic dosage form.

FIG. 3 shows a diagram of a liquid osmotic dosage form.

FIG. 4 shows a diagram of an osmotic dosage form.

FIG. 5 shows a diagram of a tri-layer osmotic dosage form.

FIG. 6 shows a diagram of an elementary osmotic pump dosage form.

FIGS. 7A-7C show diagrams of a controlled release dosage form.

FIG. 8 shows a release profile of a dosage form according to the invention;

FIG. 9 shows a plot of plasma concentration for levodopa and a levodopa complex according to the invention;

FIG. 10 shows a plot of plasma concentration for levodopa and a levodopa complex according to the invention.

### DETAILED DESCRIPTION

Definitions

The present invention is best understood by reference to the following definitions, the drawings and exemplary disclosure provided herein.

By “ascending rate of release” is meant a rate of release wherein the amount of drug released as a function of time increases over a period of time, preferably continuously and gradually. Preferably, the rate of drug released as a function of time increases in a steady (rather than step-wise) manner. More preferably, an ascending rate of release may be characterized as follows. The rate of release as a function of time for a dosage form is measured and plotted as % drug release versus time or as milligrams of drug released / hour versus time. An ascending rate of release is characterized by an average rate (expressed in mg of drug per hour) wherein the rate within a given two hour span is higher as compared with the previous two hour time span, over the period of time of about 2 hours to about 12 hours, preferably, about 2 hours to about 18 hours, more preferably about 4 hours to about 12 hours, more preferably still, about 4 hours to about 18 hours. Preferably, the increase in average rate is gradual such that less than about 30% of the dose is delivered during any 2 hour interval, more preferably, less than about 25% of the dose is delivered during any 2 hour interval. Preferably, the ascending release rate is maintained until at least about 50%, more preferably until at least about 75% of the drug in the dosage form has been released.

By “area under the curve” or “AUC” is meant the total area under the plasma drug (levodopa or carbidopa) concentration curve. It is calculated from the time of administration to the time point of the last measurable plasma drug concentration using a trapezoidal method plus an extrapolation to infinity according to the ratio of the last measurable plasma drug concentration to the apparent slope of the terminal (natural) log linear portion of the plasma drug concentration profile.

By “C” is meant the concentration of a drug in blood plasma, or serum, of a subject, generally expressed as mass per unit volume, typically nanograms per milliliter. For convenience, this concentration may be referred to herein as “drug plasma concentration”, “plasma drug concentration” or “plasma concentration” which is intended to be inclusive of drug concentration measured in any appropriate body fluid or tissue. The plasma drug concentration at any time following drug administration is referenced as Ctime, as in C9h or C24h, etc.

By “composition” is meant a drug in combination with additional active pharmaceutical ingredients, and optionally in combination with inactive ingredients, such as pharmaceutically-acceptable carriers, excipients, suspension agents, surfactants, disintegrants, binders, diluents, lubricants, stabilizers, antioxidants, osmotic agents, colorants, plasticizers, and the like.

By “complex” is meant a substance comprising a drug moiety and a transport moiety associated by a tight-ion pair bond. A drug-moiety-transport moiety complex can be distinguished from a loose ion pair of the drug moiety and the transport moiety by a difference in octanol/water partitioning behavior, characterized by the following relationship:
Δ Log D=Log D(complex)−Log D(loose-ion pair)≧0.15 (Equation 1)
wherein:
D, the distribution coefficient (apparent partition coefficient), is the ratio of the equilibrium concentrations of all species of the drug moiety and the transport moiety in octanol to the same species in water (deionized water) at a set pH (typically about pH=5.0 to about pH=7.0) at 25 degrees Celsius. Log D (complex) is determined for a complex of the drug moiety and transport moiety prepared according to the teachings herein. Log D (loose-ion pair) is determined for a physical mixture of the drug moiety and the transport moiety in deionized water. Log D can be determined experimentally or may be predicted for loose-ion pairs using commercially available software packages (e.g., ChemSilico, Inc., Advanced Chemistry Development Inc).

For instance, the octanol/water apparent partition coefficient (D=C_{octanol}/C_{water}) of a putative complex (in deionized water at 25 degree Celsius) can be determined and compared to a 1:1 (mol/mol) physical mixture of the transport moiety and the drug moiety in deionized water at 25 degree Celsius. If the difference between the Log D for the putative complex (D+T−) and the Log D for the 1:1 (mol/mol) physical mixture, D⁺μT⁻ is determined is greater than or equal to 0.15, the putative complex is confirmed as being a complex according to the invention.

In preferable embodiments, Δ Log D≧0.20, and more preferably Δ Log D≧0.25, more preferably still Δ Log D≧0.35.

By “controlled delivery ” or “controllable delivery” is meant continuous or discontinuous release of a drug over a prolonged period of time, wherein the drug is released at (a) a controlled rate over (b) a controlled period of time and in (c) a manner that provides for upper G.I. and lower G.I. tract delivery, preferably lower G.I. tract delivery, coupled with improved drug absorption as compared to the absorption of the drug in an immediate release dosage form.

Controlled delivery technologies comprise technologies that improve the upper G.I. tract and lower G.I. tract absorption of levodopa and/or carbidopa, preferably lower G.I. tract absorption of levodopa and/or carbidopa. Technologies that improve the upper G.I. tract and lower G.I. tract absorption of levodopa and/or carbidopa, preferably lower G.I. tract absorption of levodopa and/or carbidopa include, but are not limited to, (i) complexation of forms of levodopa and/or carbidopa with transport moieties and/or delivery of such complexes to the upper and lower G.I. tract, preferably the lower G.I. tract; and (ii) forming prodrugs of forms of levodopa and/or carbidopa with improved upper and lower G.I. tract, preferably lower G.I. tract, absorption and/or delivery of such prodrugs to the upper and lower G.I. tract, preferably the lower G.I. tract. In a preferred embodiment, levodopa and carbidopa are controllably delivered by complexation of levodopa and carbidopa with alkyl sulfate salts coupled with delivery of such complexes to the upper and lower G.I. tract.

By “dosage form” is meant a pharmaceutical composition in a medium, carrier, vehicle, or device suitable for administration to a patient in need thereof.

By “drug” or “drug moiety” is meant a drug, compound, or agent, or a residue of such a drug, compound, or agent that provides some pharmacological effect when administered to a subject. For use in forming a complex, the drug comprises a(n) acidic, basic, or zwitterionic structural element, or a(n) acidic, basic, or zwitterionic residual structural element. In embodiments according to the invention, drug moieties that comprise acidic structural elements or acidic residual structural elements are complexed with transport moieties that comprise basic structural elements or basic residual structural elements. In embodiments according to the invention, drug moieties that comprise basic structural elements or basic residual structural elements are complexed with transport moieties that comprise acidic structural elements or acidic residual structural elements. In embodiments according to the invention, drug moieties that comprise zwitterionic structural elements or zwitterionic residual structural elements are complexed with transport moieties that comprise either acidic or basic structural elements, or acidic or basic residual structural elements. In an embodiment, the pKa of an acidic structural element or acidic residual structural element is less than about 7.0, preferably less than about 6.0. In an embodiment, the pKa of a basic structural element or basic residual structural element is greater than about 7.0, preferably greater than about 8.0. Zwitterionic structural elements or zwitterionic residual structural elements are analyzed in terms of their individual basic structural element or basic residual structural element or their acidic structural element or acidic residual structural element, depending upon how the complex with the transport moiety is to be formed.

By “orifice” or “exit orifice” is meant means suitable for releasing the active agent from the dosage form. The expression includes aperture, hole, bore, pore, porous element, porous overlay, porous insert, hollow fiber, capillary tube, microporous insert, microporous overlay, and the like.

By “fatty acid” is meant any of the group of organic acids of the general formula CH₃(CₙHₓ)COOH where the hydrocarbon chain is either saturated (x=2n, e.g. palmitic acid, CH₃C₁₄H₂₈COOH) or unsaturated (for monounsaturated, x=2n−2, e.g. oleic acid, CH₃C₁₆H₃₀COOH).

By “immediate-release” is meant a dose of a drug that is substantially completely released from a dosage form within a time period of about 1 hour or less and, preferably, about 30 minutes or less. Certain controlled delivery dosage forms may require a short time period following administration in which to begin to release drug. In embodiments, wherein the slight delay in initial drug release is not desirable, an immediate-release overcoat can be applied to the surface of the controlled delivery dosage form. An immediate-release dose of drug applied as a coating on the surface of a dosage form refers to a dose of drug prepared in a suitable pharmaceutically acceptable carrier to form a coating solution that will dissolve rapidly upon administration thereby providing an immediate-release dose of drug. As is known in the art, such immediate release drug overcoats can contain the same or a different drug or drugs as is contained within the underlying dosage form.

By “intestine” or “gastrointestinal (G.I.) tract” is meant the portion of the digestive tract that extends from the lower opening of the stomach to the anus, composed of the small intestine (duodenum, jejunum, and ileum) and the large intestine (ascending colon, transverse colon, descending colon, sigmoid colon, and rectum).

By “loose ion-pair” is meant a pair of ions that, at physiologic pH and in an aqueous environment, are readily interchangeable with other loosely paired or free ions that may be present in the environment of the loose ion pair. Loose ion-pairs can be found experimentally by noting interchange of a member of a loose ion-pair with another ion, at physiologic pH and in an aqueous environment, using isotopic labeling and NMR or mass spectroscopy. Loose ion-pairs also can be found experimentally by noting separation of the ion-pair, at physiologic pH and in an aqueous environment, using reverse phase HPLC. Loose ion-pairs may also be referred to as “physical mixtures,” and are formed by physically mixing the ion-pair together in a medium.

By “lower gastrointestinal tract” or “lower G.I. tract” is meant the large intestine.

By “patient” is meant an animal, preferably a mammal, more preferably a human, in need of therapeutic intervention.

By “pharmaceutically acceptable salt” is meant any salt of a low solubility and/or low dissolution rate free acid pharmaceutical agent whose cation does not contribute significantly to the toxicity or pharmacological activity of the salt, and, as such, they are the pharmacological equivalents of the low solubility and/or low dissolution rate free acid pharmaceutical agent. Suitable pharmaceutically acceptable salts include base addition salts, including alkali metal salts, e.g., sodium or potassium salts; alkaline earth metal salts, e.g., calcium or magnesium salts; and salts formed with suitable organic ligands, e.g., quaternary ammonium salts, which may be similarly prepared by reacting the drug compound with a suitable pharmaceutically acceptable base.

By “pharmaceutical composition” is meant a composition suitable for administration to a patient in need thereof.

By “prolonged period of time” is meant a continuous period of time of greater than about 1 hour, preferably, greater than about 4 hours, more preferably, greater than about 8 hours, more preferably greater than about 10 hours, more preferably still, greater than about 14 hours, most preferably, greater than about 14 hours and up to about 24 hours.

As used herein, unless otherwise noted, “rate of release” or “release rate” of a drug refers to the quantity of drug released from a dosage form per unit time, e.g., milligrams of drug released per hour (mg/hr). Drug release rates for dosage forms are typically measured as an in vitro rate of drug release, i.e., a quantity of drug released from the dosage form per unit time measured under appropriate conditions and in a suitable fluid.

The release rates referred to herein are determined by placing a dosage form to be tested in de-ionized water in metal coil or metal cage sample holders attached to a USP Type VII bath indexer in a constant temperature water bath at 37° C. Aliquots of the release rate solutions, collected at pre-set intervals, are then injected into a chromatographic system fitted with an ultraviolet or refractive index detector to quantify the amounts of drug released during the testing intervals.

An alternative release rate test method may performed using the Distek 5100 (USP apparatus 2 paddle tester) in 900 mL artificial gastric fluid (AGF, pH=1.2). The temperature of the dissolution medium is maintained at 37° C. and the paddle speed is 100 rpm. The concentration of levodopa was measured with online UV spectroscopy at 280 nm.

As used herein a drug release rate obtained at a specified time refers to the in vitro release rate obtained at the specified time following implementation of the release rate test. The time at which a specified percentage of the drug within a dosage form has been released from said dosage form is referred to as the “Tx” value, where “x” is the percent of drug that has been released. For example, a commonly used reference measurement for evaluating drug release from dosage forms is the time at which 70% of drug within the dosage form has been released. This measurement is referred to as the “T70” for the dosage form. Preferably, T70 is greater than or equal to about 8 hours, more preferably, T70 is greater than or equal to about 12 hours, more preferably still, T70 is greater than to equal to about 16 hours, most preferably, T70 is greater than or equal to about 20 hours. In one embodiment, T70 is greater than or equal to about 12 hours and less than about 24 hours. In another embodiment, T70 is greater than or equal to about 8 hours and less than about 16 hours.

By “residual structural element” is meant a structural element that is modified by interaction or reaction with another compound, chemical group, ion, atom, or the like. For example, a carboxyl structural element (COOH) interacts with sodium to form a sodium-carboxylate salt, the COO— being a residual structural element.

By “solvent(s)” is meant a substance in which various other substances may be fully or partially dissolved. In the present invention, preferred solvents include aqueous solvents, and solvents having a dielectric constant less than that of water. Preferred solvents having a dielectric constant less than that of water. The dielectric constant is a measure of the polarity of a solvent and dielectric constants for exemplary solvents are shown in Table 1.

TABLE 1
Characteristics of Exemplary Solvents

| Solvent | Boiling Pt., ° C. | Dielectric constant |
|---|---|---|
| Water | 100 | 80 |
| Methanol | 68 | 33 |
| Ethanol | 78 | 24.3 |
| 1-propanol | 97 | 20.1 |
| 1-butanol | 118 | 17.8 |
| acetic acid | 118 | 6.15 |
| Acetone | 56 | 20.7 |
| methyl ethyl ketone | 80 | 18.5 |
| ethyl acetate | 78 | 6.02 |
| Acetonitrile | 81 | 36.6 |
| N,N-dimethylformamide (DMF) | 153 | 38.3 |
| diemthyl sulfoxide (DMSO) | 189 | 47.2 |
| Hexane | 69 | 2.02 |
| Benzene | 80 | 2.28 |
| diethyl ether | 35 | 4.34 |
| tetrahydrofuran (THF) | 66 | 7.52 |
| methylene chloride | 40 | 9.08 |
| carbon tetrachloride | 76 | 2.24 |

The solvents water, methanol, ethanol, 1-propanol, 1-butanol, and acetic acid are polar protic solvents having a hydrogen atom attached to an electronegative atom, typically oxygen. The solvents acetone, ethyl acetate, methyl ethyl ketone, and acetonitrile are dipolar aprotic solvents, and are in one embodiment, preferred for use in forming the inventive complexes. Dipolar aprotic solvents do not contain an OH bond but typically have a large bond dipole by virtue of a multiple bond between carbon and either oxygen or nitrogen. Most dipolar aprotic solvents contain a C—O double bond. Solvents having a dielectric constant less than that of water are particularly useful in the formation of the inventive complexes. The dipolar aprotic solvents noted in Table 1 have a dielectric constant at least two-fold lower than water and a dipole moment close to or greater than water.

By “structural element” is meant a chemical group that (i) is part of a larger molecule, and (ii) possesses distinguishable chemical functionality. For example, an acidic group or a basic group on a compound is a structural element.

By “substance” is meant a chemical entity having specific characteristics.

By “tight-ion pair” is meant a pair of ions that are, at physiologic pH and in an aqueous environment are not readily interchangeable with other loosely paired or free ions that may be present in the environment of the tight-ion pair. A tight-ion pair can be experimentally detected by noting the absence of interchange of a member of a tight ion-pair with another ion, at physiologic pH and in an aqueous environment, using isotopic labeling and NMR or mass spectroscopy. Tight ion pairs also can be found experimentally by noting the lack of separation of the ion-pair, at physiologic pH and in an aqueous environment, using reverse phase HPLC.

By “therapeutically effective amount” is meant that amount of a drug that elicits the biological or medicinal response in a tissue system, animal or human that is being sought by a researcher, veterinarian, medical doctor or other clinician, which includes alleviation of the symptoms of the disease or disorder being treated. More specifically, a therapeutically effective amount of the inventive substances preferably alleviates symptoms, complications, or biochemical indicia of diseases responsive to levodopa or carbidopa therapy. The exact dose will be ascertainable by one skilled in the art using known techniques (see, e.g., Lieberman, Pharmaceutical Dosage Forms (Vols. 1-3, 1992); Lloyd, 1999, The Art, Science, and Technology of Pharmaceutical Compounding; and Pickar, 1999, Dosage Calculations). A therapeutically effective dose is also one in which any toxic or detrimental side effects of the active agent is outweighed in clinical terms by therapeutically beneficial effects. It is to be further noted that for each particular subject, specific dosage regimens should be evaluated and adjusted over time according to the individual need and professional judgment of the person administering or supervising the administration of the compounds.

By “transport moiety” is meant a compound that is capable of forming, or a residue of that compound that has formed, a complex with a drug, wherein the transport moiety serves to improve transport of the drug across epithelial tissue, compared to that of the uncomplexed drug. The transport moiety comprises a hydrophobic portion and a(n) acidic, basic, or zwitterionic structural element, or a(n) acidic, basic, or zwitterionic residual structural element. In a preferred embodiment, the hydrophobic portion comprises a hydrocarbon chain. In an embodiment, the pKa of a basic structural element or basic residual structural element is greater than about 7.0, preferably greater than about 8.0. Zwitterionic structural elements or zwitterionic residual structural elements are analyzed in terms of their individual basic structural element or basic residual structural element or their acidic structural element or acidic residual structural element, depending upon how the complex with the drug moiety is to be formed.

In a more preferred embodiment, transport moieties comprise pharmaceutically acceptable acids, including but not limited to carboxylic acids, and salts thereof. In embodiments, transport moieties comprise fatty acids or its salts, benzenesulfonic acid or its salts, benzoic acid or its salts, fumaric acid or its salts, or salicylic acid or its salts. In preferred embodiments the fatty acids or their salts, comprise from 6 to 18 carbon atoms (C6-C18), more preferably 8 to 16 carbon atoms (C8-C16), even more preferably 10 to 14 carbon atoms (C10-C14), and most preferably 12 carbon atoms (C12).

In more preferred embodiments, transport moieties comprise alkyl sulfates (either saturated or unsaturated) and their salts, such as potassium, magnesium, and sodium salts, including particularly sodium octyl sulfate, sodium decyl sulfate, sodium lauryl sulfate, and sodium tetradecyl sulfate. In preferred embodiments the alkyl sulfate or its salt comprise from 6 to 18 carbon atoms (C6-C18), more preferably 8 to 16 carbon atoms (C8-C16), even more preferably 10 to 14 carbon atoms (C10-C14), and most preferably 12 carbon atoms (C12). Also suitable are other anionic surfactants.

In another more preferred embodiment, transport moieties comprise pharmaceutically acceptable primary amines or salts thereof, particularly primary aliphatic amines (both saturated and unsaturated) or salts thereof, diethanolamine, ethylenediamine, procaine, choline, tromethamine, meglumine, magnesium, aluminum, calcium, zinc, alkyltrimethylammonium hydroxides, alkyltrimethylammonium bromides, benzalkonium chloride and benzethonium chloride. Also useful are other pharmaceutically acceptable compounds that comprise secondary or tertiary amines, and their salts, and cationic surfactants.

By “upper gastrointestinal tract” or “upper G.I. tract” is meant that portion of the gastrointestinal tract including the stomach and the small intestine.

By “window” is meant a period of time having a defined duration. Windows preferably begin at time of administration of a dosage form to a patient, or any time thereafter. For instance, in an embodiment a window may have a duration of about 12 hours. In a preferable embodiments, the window may begin at a variety of times. For instance, in a preferable embodiment, the window may begin about 1 hour after administration of a dosage form, and have a duration of about 12 hours, which means that the window would open about 1 hour after administration of the dosage from and close at about 13 hours following administration of the dosage form.

By “zero order rate of release” is meant a rate of release wherein the amount of drug released as a function of time is substantially constant. More particularly, the rate of release of drug as a function of time shall vary by less than about 30%, preferably, less than about 20%, more preferably, less than about 10%, most preferably, less than about 5%, wherein the measurement is taken over the period of time wherein the cumulative release is between about 25% and about 75%, preferably, between about 25% and about 90% by total weight of drug in the dosage form.

By “zero order plasma profile” is meant a substantially flat or unchanging amount of a particular drug in the plasma of a patient over a particular time interval. Generally, the plasma concentration of a drug exhibiting a zero order plasma profile will vary by no more than about 30% and preferably by no more than about 10% from one time interval to the subsequent time interval.

### Controlled Delivery, Complex Formation, and Characterization

The inventors have unexpectedly discovered that it is possible to solve the problems in the art discussed above using substances, compositions, dosage forms and methods that deliver levodopa and/or carbidopa using controlled delivery approaches as set forth herein. Such substances, compositions, dosage forms and methods are useful, inter alia, in the treatment of Parkinson's disease.

In particular, the inventors note that a conventional solution to the aforementioned problems might be application of conventional controlled release technologies. However, upon close examination of the data, the inventors have discovered that these conventional controlled release technologies are insufficient to solve the aforementioned problems in the art.

As a pharmacokinetic understanding, the inventors have recognized that levodopa and/or carbidopa is poorly absorbed in the lower G.I. tract, and possibly even in portions of the upper G.I. tract. This is borne out by the understanding in the art that levodopa is transported across the intestinal epithelium primarily by carriers for large neutral L-amino acids. D. Nilsson et al., “Absorption of L-DOPA from the proximal small intestine studied in the rhesus monkey by positron emission tomography”, European J. of Pharm. Sci. 7:185-189 (1999). These carriers are known to be concentrated in portions of the proximal upper G.I. tract. Additionally, absorption studies have shown that practically all absorption of levodopa occurs within 4 hours after oral administration, indicating that almost all absorption occurs in the upper G.I. tract. The authors concluded “These data suggest that the release of levodopa cannot be sustained beyond that of Sinemet CR without further reduction in bioavailability and increase in variability.” I. R. Wilding et al., “Characterisation of the In Vivo Behaviour of a Controlled-Release Formulation of Levodopa (Sinemet CR)”, Clin. Neuropharmacology 14(4):305-321 (1991).

While data regarding intestinal absorption of carbidopa is fairly scarce, the inventors have recognized that its absorption may also be concentrated in the upper G.I. tract, as its structure lends itself to being transported by the same amino acid transporters as are hypothesized to transport levodopa. Accordingly, fluctuating carbidopa levels, due to poor absorption of carbidopa in portions of the G.I. tract, can lead to periods wherein the plasma concentration of carbidopa is significantly reduced with concomitant periods of lower levodopa levels (due to metabolism of levodopa). These fluctuations are undesirable, as noted elsewhere herein.

The inventors have further recognized that poor lower G.I. tract (and portions of the distal upper G.I. tract) absorption implies that conventional controlled release (CR) techniques will not work in the development of an oral dosage form of levodopa and/or carbidopa that exhibits reduced concentration fluctuations. In particular, such techniques cannot be used to develop substantially zero order plasma profiles, preferably zero order plasma profiles, such as are produced with intraduodenal infusion. Generally, a CR dosage form will move through the upper G.I. tract to the lower G.I. tract within 8-10 hours or less. Once the dosage form that included levodopa and/or carbidopa arrived at the lower G.I. tract, absorption of the compound would be significantly reduced. In fact, as noted above, absorption is essentially complete as quickly as 4 hours after dosing. Therefore, CR dosage forms would have to be dosed more frequently than bid or qd to achieve efficacy. This may be the source of the undesirable concentration fluctuations and peak effects noted above.

Accordingly, the inventors have surprisingly recognized that only a specific sub-class of controlled release technologies, referred to herein as controlled delivery technologies, would suffice to provide bid or qd dosing of levodopa and/or carbidopa.

These controlled delivery technologies comprise substances comprising levodopa and/or substances comprising carbidopa that demonstrate improved lower G.I. tract absorption. Substances comprising levodopa and/or substances comprising carbidopa that demonstrate improved lower G.I. tract absorption include, but are not limited to, complexes of levodopa and/or carbidopa with alkyl sulfate salts; and prodrugs of levodopa and/or carbidopa possessing improved lower G.I. absorption. In a preferred embodiment, levodopa and/or carbidopa in the form of a complex is controllably delivered to a patient in need thereof.

Controlled delivery of substances comprising levodopa and/or substances comprising carbidopa, according to the present invention, provides a mechanism whereby substantially zero order plasma profiles, preferably zero order plasma profiles, of levodopa and/or carbidopa may be achieved. Such substantially zero order plasma profiles, preferably zero order plasma profiles, may alleviate the problems noted in the prior art with regards to oral dosage forms (concentration swings and peak effects), while providing substantially improved convenience of dosing as compared to infusion pumps.

Various embodiments of the inventive controlled delivery technologies will now be discussed further herein.

In certain embodiments, levodopa and/or carbidopa are/is modified so as to demonstrate improved lower G.I. tract absorption. Pharmaceutical development typically targets drug forms for absorption in the upper G.I. tract instead of the lower G.I. tract because the upper G.I. tract has a far greater surface area for absorption of drugs than does the lower G.I. tract. The lower G.I. tract lacks microvilli which are present in the upper G.I. tract. The presence of microvilli greatly increases the surface area for drug absorption, and the upper G.I. tract has 480 times the surface area than does the lower G.I. tract. Differences in the cellular characteristics of the upper and lower G.I. tracts also contribute to the poor absorption of molecules in the lower G.I tract.

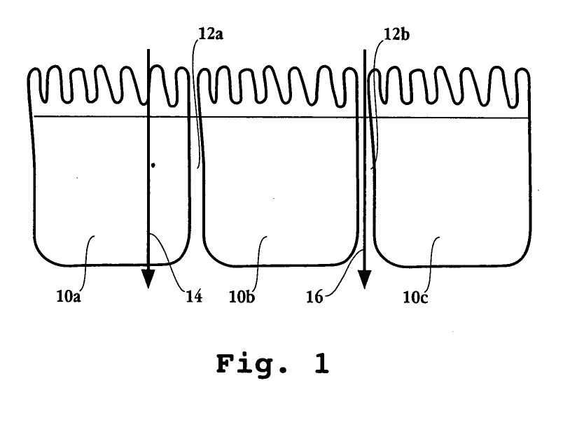

FIG. 1 illustrates two common routes for transport of compounds across the epithelium of the G.I. tract. Individual epithelial cells, represented by 10a, 10b, 10c, form a cellular barrier along the small and large intestine. Individual cells are separated by water channels or tight junctions, such as junctions 12a, 12b. Transport across the epithelium occurs via either or both a transcellular pathway and a paracellular pathway. The transcellular pathway for transport, indicated in FIG. 1 by arrow 14, involves movement of the compound across the wall and body of the epithelial cell by passive diffusion or by carrier-mediated transport. The paracellular pathway of transport involves movement of molecules through the tight junctions between individual cells, as indicated by arrow 16. Paracellular transport is less specific but has a much greater overall capacity, in part because it takes place throughout the length of the G.I. tract. However, the tight junctions vary along the length of the G.I. tract, with an increasing proximal to distal gradient in effective ‘tightness’ of the tight junction. Thus, the duodenum in the upper G.I. tract is more “leaky” than the ileum in the upper G.I. tract which is more “leaky” than the colon, in the lower G.I. tract (Knauf, H. et al., Klin. Wochenschr., 60(19):1191-1200 (1982)).

Since the typical residence time of a drug in the upper G.I. tract is from approximately four to six hours, drugs having poor lower G.I. absorption are absorbed by the body through a period of only four to six hours after oral ingestion. Frequently it is medically desirable that the administered drug be presented in the patient's blood stream at a relatively constant concentration throughout the day. To achieve this with traditional drug formulations that exhibit minimal lower G.I. tract absorption, patients would need to ingest the drugs three to four times a day. Practical experience with this inconvenience to patients suggests that this is not an optimum treatment protocol. The situation with levodopa and/or carbidopa is one example.

To provide constant dosing treatments, conventional pharmaceutical development has suggested various controlled release drug systems. Such systems function by releasing their payload of drugs over an extended period of time following administration. However, these conventional forms of controlled release systems are not effective in the case of drugs exhibiting minimal colonic absorption. Since the drugs are only absorbed in the upper G.I. tract and since the residence time of the drug in the upper G.I. tract is only four to six hours, the fact that a proposed controlled release dosage form may release its payload after the residence period of the dosage form in the upper G.I. does not mean the that body will continue to absorb the controlled release drug past the four to six hours of upper G.I. tract residence. Instead, the drug released by the controlled release dosage form after the dosage form has entered the lower G.I. tract is generally not absorbed and, instead, is expelled from the body.

It has been surprisingly found that many common drug moieties with poor absorption characteristics, once complexed with certain transport moieties, exhibit significantly enhanced absorption, particularly lower G.I. tract absorption although upper GI tract absorption may also be enhanced. It is further surprising that complexes, such as certain substances comprising levodopa and/or substances comprising carbidopa, according to the invention show improved absorption as compared to loose ion-pairs (i.e. a non-complexed form) that comprise the same ions as the inventive complexes.

These unexpected results have been found to apply to many categories of drug moieties, including drug moieties that comprise a basic structural element or a basic residual structural element. The unexpected results of the present invention also apply to drug moieties that comprise a zwitterionic structural element or a zwitterionic residual structural element. An example of such a drug moiety comprises levodopa and/or carbidopa. The unexpected results of the present invention also apply to drug moieties that comprise an acidic structural element or an acidic residual structural element.

While not wishing to be bound by specific understanding of mechanisms, the inventors reason as follows:

When loose ion-pairs are placed in a polar solvent environment, it is assumed that polar solvent molecules will insert themselves in the space occupied by the ionic bond, thus driving apart the bound ions. A solvation shell, comprising polar solvent molecules electrostatically bonded to a free ion, may be formed around the free ion. This solvation shell then prevents the free ion from forming anything but a loose ion-pairing ionic bond with another free ion. In a situation wherein there are multiple types of counter ions present in the polar solvent, any given loose ion-pairing may be relatively susceptible to counter-ion competition.

This effect is more pronounced as the polarity, expressed as the dielectric constant of the solvent, increases. Based on Coulomb's law, the force between two ions with charges (q1) and (q2) and separated by a distance( r) in a medium of dielectric constant (e) is:

$$F = - \frac{{q}_{1} {q}_{2}}{4 {πɛ}_{0} ɛ {r}^{2}} \qquad ( Equation 2 )$$

where ε₀ is the constant of permittivity of space. The equation shows the importance of dielectric constant (ε) on the stability of a loose ion-pair in solution. In aqueous solution that has a high dielectric constant (ε=80), the electrostatic attraction force is significantly reduced if water molecules attack the ionic bonding and separate the opposite charged ions.

Therefore, high dielectric constant solvent molecules, once present in the vicinity of the ionic bond, will attack the bond and eventually break it. The unbound ions then are free to move around in the solvent. These properties characterize a loose ion-pair.

Tight ion-pairs are formed differently from loose-ion pairs, and consequently possess different properties from a loose ion-pair. Tight ion-pairs are formed by reducing the number of polar solvent molecules in the bond space between two ions. This allows the ions to move tightly together, and results in a bond that is significantly stronger than a loose ion-pair bond, but is still considered an ionic bond. As disclosed more fully herein, tight ion-pairs are obtained using less polar solvents than water so as to reduce entrapment of polar solvents between the ions.

For additional discussion of loose and tight ion-pairs, see D. Quintanar-Guerrero et al., “Applications of the Ion Pair Concept to Hydrophilic Substances with Special Emphasis on Peptides,” Pharm. Res. 14(2):119-127 (1997).

The difference between loose and tight ion-pairing also can be observed using chromatographic methods. Using reverse phase chromatography, loose ion-pairs can be readily separated under conditions that will not separate tight ion-pairs.

Bonds according to this invention may also be made stronger by selecting the strength of the cation and anion relative to one another. For instance, in the case where the solvent is water, the cation (base) and anion (acid) can be selected to attract one another more strongly. If a weaker bond is desired, then weaker attraction may be selected.

Portions of biological membranes can be modeled to a first order approximation as lipid bilayers for purposes of understanding molecular transport across such membranes. Transport across the lipid bilayer portions (as opposed to active transporters, etc.) is unfavorable for ions because of unfavorable portioning. Various researchers have proposed that charge neutralization of such ions can enhance cross-membrane transport.

In the “ion-pair” theory, ionic drug moieties are paired with transport moiety counter ions to “bury” the charge and render the resulting ion-pair more liable to move through a lipid bilayer. This approach has generated a fair amount of attention and research, especially with regards to enhancing absorption of orally administered drugs across the intestinal epithelium.

While ion-pairing has generated a lot of attention and research, it has not always generated a lot of success. For instance, ion-pairs of two antiviral compounds were found not to result in increased absorption due to the effects of the ion-pair on trans-cellular transport, but rather to an effect on monolayer integrity. The authors concluded that the formation of ion pairs may not be very efficient as a strategy to enhance transepithelial transport of charged hydrophilic compounds as competition by other ions found in in vivo systems may abolish the beneficial effect of counter-ions. J. Van Gelder et al., “Evaluation of the Potential of Ion Pair Formation to Improve the Oral Absorption of two Potent Antiviral Compounds, AMD3 100 and PMPA”, Int. J. of Pharmaceutics 186:127-136 (1999). Other authors have noted that absorption experiments with ion-pairs have not always pointed at clear-cut mechanisms. D. Quintanar-Guerrero et al., Applications of the Ion Pair Concept to Hydrophilic Substances with Special Emphasis on Peptides, Pharm. Res. 14(2):119-127 (1997).

The inventors have unexpectedly discovered that a problem with these ion-pair absorption experiments is that they were performed using loose-ion pairs, rather than tight ion-pairs. Indeed, many ion-pair absorption experiments disclosed in the art do not even expressly differentiate between loose ion-pairs and tight ion-pairs. One of skill has to distinguish that loose ion-pairs are disclosed by actually reviewing the disclosed methods of making the ion-pairs and noting that such disclosed methods of making are directed to loose ion-pairs not tight ion-pairs. Loose ion-pairs are relatively susceptible to counter-ion competition, and to solvent-mediated (e.g. water-mediated) cleavage of the ionic bonds that bind loose ion-pairs. Accordingly, when the drug moiety of the ion-pair arrives at an intestinal epithelial cell membrane wall, it may or may not be associated in a loose ion-pair with a transport moiety. The chances of the ion-pair existing near the membrane wall may depend more on the local concentration of the two individual ions than on the ion bond keeping the ions together. Absent the two moieties being bound when they approached an intestinal epithelial cell membrane wall, the rate of absorption of the non-complexed drug moiety might be unaffected by the non-complexed transport moiety. Therefore, loose ion-pairs might have only a limited impact on absorption compared to administration of the drug moiety alone.

In contrast, the inventive complexes possess bonds that are more stable in the presence of polar solvents such as water. Accordingly, the inventors reasoned that, by forming a complex, the drug moiety and the transport moiety would be more likely to be associated as ion-pairs at the time that the moieties would be near the membrane wall. This association would increase the chances that the charges of the moieties would be buried and render the resulting ion-pair more liable to move through the cell membrane.

In an embodiment, the complex comprises a tight ion-pair bond between the drug moiety and the transport moiety. As discussed herein, tight ion-pair bonds are more stable than loose ion-pair bonds, thus increasing the likelihood that the drug moiety and the transport moiety would be associated as ion-pairs at the time that the moieties would be near the membrane wall. This association would increase the chances that the charges of the moieties would be buried and render the tight ion-pair bound complex more liable to move through the cell membrane.

It should be noted that the inventive complexes may improve absorption relative to the non-complexed drug moiety throughout the G.I. tract, not just the lower G.I. tract, as the complex is intended to improve transcellular transport generally, not just in the lower G.I. tract. For instance, if the drug moiety is a substrate for an active transporter found primarily in the upper G.I., the complex formed from the drug moiety may still be a substrate for that transporter. Accordingly, the total transport may be a sum of the transport flux effected by the transporter plus the improved transcellular transport provided by the present invention. In an embodiment, the inventive complex provides improved absorption in the upper G.I. tract, the lower G.I. tract, and both the upper G.I. tract and the lower G.I. tract.

Complexes according to the invention can be made up of a variety of drug and transport moieties. Generally speaking, the drug moiety is selected first, and then the appropriate transport moiety is selected to form the inventive complex. One of skill could consider a number of factors in selecting transport moieties, including but not limited to the toxicity and tolerability of the transport moiety, the polarity of the structural element or structural element residue of the drug moiety, the strength of the structural element or structural element residue of the drug moiety, the strength of the structural element or structural element residue of the transport moiety, possible therapeutic advantages of the transport moiety. In certain preferred embodiments, the hydrophobic portions of the transport moiety comprises a hydrophobic chain, more preferably an alkyl chain. This alkyl chain may help to promote stability of the complex through sterically protecting the ionic bond from attack by polar solvent molecules.

It should be noted that the inventive complexes may improve absorption relative to the non-complexed drug moiety throughout the G.I. tract, not just the lower G.I. tract, as the complex is intended to improve transcellular transport generally, not just in the lower G.I. tract. For instance, if the drug moiety is a substrate for an active transporter found primarily in the upper G.I., the complex formed from the drug moiety may still be a substrate for that transporter. Accordingly, the total transport may be a sum of the transport flux effected by the transporter plus the improved transcellular transport provided by the present invention. In an embodiment, the inventive complex provides improved absorption in the upper G.I. tract, the lower G.I. tract, and both the upper G.I. tract and the lower G.I. tract.

Complexes according to the invention can be made up of a variety of drug and transport moieties. Generally speaking, the drug moiety is selected first, and then the appropriate transport moiety is selected to form the inventive complex. One of skill could consider a number of factors in selecting transport moieties, including but not limited to the toxicity and tolerability of the transport moiety, the polarity of the structural element or structural element residue of the drug moiety, the strength of the structural element or structural element residue of the drug moiety, the strength of the structural element or structural element residue of the transport moiety, possible therapeutic advantages of the transport moiety, and the steric hindrance of the bond between the drug moiety and the transport moiety that is provided by the transport moiety.

In preferred embodiments the transport moieties comprise alkyl sulfates or their salts, having from 6 to 18 carbon atoms (C6-C18), more preferably 8 to 16 carbon atoms (C8-C16), even more preferably 10 to 14 carbon atoms (C10-C14), and most preferably 12 carbon atoms (C12). In other preferred embodiments, the transport moieties comprise fatty acids, or their salts, having from 6 to 18 carbon atoms (C6-C18), more preferably 8 to 16 carbon atoms (C8-C16), even more preferably 10 to 14 carbon atoms (C10-C14), and most preferably 12 carbon atoms (C12). Methods of making the inventive 3ANBPA complexes are disclosed herein, including the appended Examples.

An alternative manner of improving lower G.I. absorption of levodopa and carbidopa is to produce prodrugs of the compounds that are substrates for active transporters expressed in epithelial cells lining the lumen of the human colon. U.S. patent application 20030158254 to Zerangue et al., filed Aug. 21, 2003, entitled “Engineering absorption of therapeutic compounds via colonic transporters” (“Zerangue”), hereby incorporated by reference in its entirety for all purposes, discloses drugs modified to be such substrates, including compounds suitable for use in extended release oral dosage forms, particularly those that release drug over periods of greater than about 2-4 hours following administration.

Zerangue discloses a variety of transporters useful in the practice of this invention, comprising the sodium dependent multi-vitamin transporter (SMVT), and monocarboxylate transporters 1 and 4 (MCT 1 and MCT 4). Zerangue also discloses methods of identifying agents or conjugate moieties that are substrates of a transporter, and agents, conjugates, and conjugate moieties that can be screened. In particular, Zerangue discloses compounds to be screened that are variants of known transporter substrates. Such compounds comprise bile salts or acids, steroids, ecosanoids, or natural toxins or analogs thereof, as described by Smith, Am. J. Physiol. 2230, 974-978 (1987); Smith, Am. J. Physiol. 252, G479-G484 (1993); Boyer, Proc. Natl. Acad. Sci. USA 90, 435-438 (1993); Fricker, Biochem. J. 299, 665-670 (1994); Ficker, Biochem J. 299, 665-670 (1994); Ballatori, Am. J. Physiol. 278. Zerangue further discloses the linkage of agents to conjugate moieties, and several prodrugs, comprising pivaloxymethyl gabaptentin carbamate, gabapentin acetoxyethyl carbamate, and alpha-aminopropylisobutyryl gabapentin. Levodopa and carbidopa are disclosed at paragraph 92 of Zerangue.

Prodrugs of levodopa and carbidopa that are substrates for active transporters expressed in epithelial cells lining the lumen of the human colon are specifically encompassed by the present invention. The prodrugs of levodopa and carbidopa can be delivered using the controlled delivery technologies disclosed herein.

### Exemplary Dosage Forms and Methods of Use

A variety of dosage forms are suitable for use with the drugs of interest. In embodiments, dosage forms that permit dosing that maintains a plasma drug concentration that is at least about fifteen percent of the Cmax throughout a window of at least about ten hours duration are provided. A dosage form may be configured and formulated according to any design that delivers a desired dose of levodopa or carbidopa. In certain embodiments, the dosage form is orally administrable and is sized and shaped as a conventional tablet or capsule. Orally administrable dosage forms may be manufactured according to one of various different approaches. For example, the dosage form may be manufactured as a diffusion system, such as a reservoir device or matrix device, a dissolution system, such as encapsulated dissolution systems (including, for example, “tiny time pills”, and beads) and matrix dissolution systems, and combination diffusion/dissolution systems and ion-exchange resin systems, as described in Remington's Pharmaceutical Sciences, 18th Ed., pp. 1682-1685 (1990).

One important consideration in the practice of this invention is the physical state of the drug substance to be delivered by the dosage form. In certain embodiments, substances comprising levodopa and/or substances comprising carbidopa may be in a paste or liquid state, inc which case solid dosage forms may not be suitable for use in the practice of this invention. In such cases, dosage forms capable of delivering substances in a paste or liquid state should be used. For instance, an inventive complex comprising levodopa and sodium lauryl sulfate may be in a paste-like state. In such case, dosage forms capable of delivering substances in a paste or liquid state should be used to deliver the complex. Alternatively, in certain embodiments, a different transport moiety may be used to raise the melting point of the substances, thus making it more likely that the inventive complexes will be present in a solid form.

A specific example of a dosage form suitable for use with the present invention is an osmotic dosage form. Osmotic dosage forms, in general, utilize osmotic pressure to generate a driving force for imbibing fluid into a compartment formed, at least in part, by a semipermeable wall that permits free diffusion of fluid but not drug or osmotic agent(s), if present. An advantage to osmotic systems is that their operation is pH-independent and, thus, continues at the osmotically determined rate throughout an extended time period even as the dosage form transits the gastrointestinal tract and encounters differing microenvironments having significantly different pH values. A review of such dosage forms is found in Santus and Baker, “Osmotic drug delivery: a review of the patent literature,” Journal of Controlled Release, 35:1-21 (1995). Osmotic dosage forms are also described in detail in the following U.S. Patents, each incorporated in their entirety herein: U.S. Pat. Nos. 3,845,770; 3,916,899; 3,995,631; 4,008,719; 4,111,202; 4,160,020; 4,327,725; 4,519,801; 4,578,075; 4,681,583; 5,019,397; and 5,156,850.

The present invention provides a controlled delivery liquid formulation of substances that comprise levodopa and/or substances that comprise carbidopa for use with oral osmotic devices. Oral osmotic devices for delivering liquid formulations and methods of using them are known in the art, for example, as described and claimed in the following U.S. Patents owned by ALZA corporation: U.S. Pat. Nos. 6,419,952; 6,174,547; 6,551,613; 5,324,280; 4,111,201; and 6,174,547; each of which is hereby incorporated by reference in its entirety for all purposes. Methods of using oral osmotic devices for delivering therapeutic agents at an ascending rate of release can be found in International Application Numbers WO 98/06380, WO 98/23263, and WO 99/62496, each of which is hereby incorporated by reference in its entirety for all purposes.

Exemplary liquid carriers for the present invention include lipophilic solvents (e.g., oils and lipids), surfactants, and hydrophilic solvents. Exemplary lipophilic solvents, for example, include, but are not limited to, Capmul PG-8, Caprol MPGO, Capryol 90, Plurol Oleique CC497, Capmul MCM, Labrafac PG, N-Decyl Alcohol, Caprol 10G10O, Oleic Acid, Vitamin E, Maisine 35-1, Gelucire 33/01, Gelucire 44/14, Lauryl Alcohol, Captex 355EP, Captex 500, Capylic/Caplic Triglyceride, Peceol, Caprol ET, Labrafil M2125 CS, Labrafac CC, Labrafil M 1944 CS, Captex 8277, Myvacet 9-45, Isopropyl Nyristate, Caprol PGE 860, Olive Oil, Plurol Oleique, Peanut Oil, Captex 300 Low C6, and Capric Acid. Exemplary surfactants for example, include, but are not limited to, Vitamin E TPGS, Cremophor EL-P, Labrasol, Tween 20, Cremophor RH40, Pluronic L-121, Acconon S-35, Pluronic L-31, Pluronic L-35, Pluronic L-44, Tween 80, Pluronic L-64, Solutol HS-15, Span 20, Cremophor EL, Span 80, Pluronic L-43, and Tween 60. Exemplary hydrophilic solvents for example, include, but are not limited to, Isosorbide Dimethyl Ether, Polyethylene Glycol 400 (PEG-3000), Transcutol HP, Polyethylene Glycol 400 (PEG-4000), Polyethylene Glycol 400 (PEG-300), Polyethylene Glycol 400 (PEG-6000), Polyethylene Glycol 400 (PEG-400), Polyethylene Glycol 400 (PEG-8000), Polyethylene Glycol 400 (PEG-600), and Propylene Glycol (PG).

In one embodiment, a liquid formulation comprises from about 10% to about 90% of substances comprising levodopa complex, 10% to about 30% of substances comprising carbidopa, and about 10% to about 90% of one or more liquid carriers. For example, in some embodiments, the liquid formulation will comprise levodopa and a hydrophilic solvent such as PG. In such embodiments, the liquid formulation can comprise from about 10% to about 90% of substances comprising levodopa complex and about 10% to about 90% of the hydrophilic solvent. In other embodiments, the liquid formulation can comprise about 40% substances comprising levodopa complex, 10% substances comprising carbidopa complex, and about 50% liquid carrier. In one such preferred embodiment, the liquid carrier can comprise about 50% surfactant, such as Cremophor EL, solutol, or Tween 80, and about 50% hydrophilic solvent, such as PG.

The skilled practitioner will understand that any formulation comprising a sufficient dosage of substances comprising levodopa and/or substances comprising carbidopa solubilized in a liquid carrier suitable for administration to a subject and for use in an osmotic device can be used in the present invention. In one exemplary embodiment of the present invention, the liquid carrier is PG, Solutol, Cremophor EL, or a combination thereof.

The liquid formulation according to the present invention can also comprise, for example, additional excipients such as an antioxidant, permeation enhancer and the like. Antioxidants can be provided to slow or effectively stop the rate of any autoxidizable material present in the capsule. Representative antioxidants can comprise a member selected from the group of ascorbic acid; alpha tocopherol; ascorbyl palmitate; ascorbates; isoascorbates; butylated hydroxyanisole; butylated hydroxytoluene; nordihydroguiaretic acid; esters of garlic acid comprising at least 3 carbon atoms comprising a member selected from the group consisting of propyl gallate, octyl gallate, decyl gallate, decyl gallate; 6-ethoxy-2,2,4-trimethyl-1,2-dihydro-guinoline; N-acetyl-2,6-di-t-butyl-p-aminophenol; butyl tyrosine; 3-tertiarybutyl-4-hydroxyanisole; 2-tertiary-butyl-4-hydroxyanisole; 4-chloro-2,6-ditertiary butyl phenol; 2,6-ditertiary butyl p-methoxy phenol; 2,6-ditertiary butyl-p-cresol: polymeric antioxidants; trihydroxybutyro-phenone physiologically acceptable salts of ascorbic acid, erythorbic acid, and ascorbyl acetate; calcium ascorbate; sodium ascorbate; sodium bisulfite; and the like. The amount of antioxidant used for the present purposes, for example, can be about 0.001% to 25% of the total weight of the composition present in the lumen. Antioxidants are known to the prior art in U.S. Pat. Nos. 2,707,154; 3,573,936; 3,637,772; 4,038,434; 4,186,465 and 4,559,237, each of which is hereby incorporated by reference in its entirety for all purposes.

The inventive liquid formulation can comprise permeation enhancers that facilitate absorption of the active agent in the environment of use. Such enhancers can, for example, open the so-called “tight junctions” in the gastrointestinal tract or modify the effect of cellular components, such a p-glycoprotein and the like. Suitable enhancers can include alkali metal salts of salicyclic acid, such as sodium salicylate, caprylic or capric acid, such as sodium caprylate or sodium caprate, and the like. Enhancers can include, for example, the bile salts, such as sodium deoxycholate. Various p-glycoprotein modulators are described in U.S. Pat. Nos. 5,112,817 and 5,643,909, each of which is hereby incorporated by reference in its entirety for all purposes. Various other absorption enhancing compounds and materials are described in U.S. Pat. No. 5,824,638, which also is incorporated herein by reference in its entirety for all purposes. Enhancers can be used either alone or as mixtures in combination with other enhancers.

In certain embodiments, substances that comprise levodopa and/or substances that comprise are administered as a self-emulsifying formulation. Like the other liquid carriers, the surfactant functions to prevent aggregation, reduce interfacial tension between constituents, enhance the free-flow of constituents, and lessen the incidence of constituent retention in the dosage form. The therapeutic emulsion formulation of this invention comprises a surfactant that imparts emulsification. Exemplary surfactants can also include, for example, in addition to the surfactants listed above, a member selected from the group consisting of polyoxyethylenated castor oil comprising 9 moles of ethylene oxide, polyoxyethylenated castor oil comprising 15 moles of ethylene oxide, polyoxyethylene caster oil comprising 20 moles of ethylene oxide, polyoxyethylenated caster oil comprising 25 moles of ethylene oxide, polyoxyethylenated caster oil comprising 40 moles of ethylene oxide, polyoxyethylenated castor oil comprising 52 moles of ethylene oxide, polyoxyethylenated sorbitan monopalmitate comprising 20 moles of ethylene oxide, polyoxyethylenated sorbitan monostearate comprising 20 moles of ethylene oxide, polyoxyethylenated sorbitan monostearate comprising 4 moles of ethylene oxide, polyoxyethylenated sorbitan tristearate comprising 20 moles of ethylene oxide, polyoxyethylenated sorbitan monostearate comprising 20 moles of ethylene oxide, polyoxyethylenated sorbitan trioleate comprising 20 moles of ethylene oxide, polyoxyethylene lauryl ether, polyoxyethylenated stearic acid comprising 40 moles of ethylene oxide, polyoxyethylenated stearic acid comprising 50 moles of ethylene oxide, polyoxyethylenated stearyl alcohol comprising 2 moles of ethylene oxide, and polyoxyethylenated oleyl alcohol comprising 2 moles of ethylene oxide. The surfactants are available from Atlas Chemical Industries.

The drug emulsified formulations of the present invention can initially comprise an oil and a non-ionic surfactant. The oil phase of the emulsion comprises any pharmaceutically acceptable oil which is not immiscible with water. The oil can be an edible liquid such as an non-polar ester of an unsaturated fatty acid, derivatives of such esters, or mixtures of such esters. The oil can be vegetable, mineral, animal or marine in origin. Examples of non-toxic oils can also include, for example, in addition to the surfactants listed above, a member selected from the group consisting of peanut oil, cottonseed oil, sesame oil, corn oil, almond oil, mineral oil, castor oil, coconut oil, palm oil, cocoa butter, safflower, a mixture of mono- and diglycerides of 16 to 18 carbon atoms, unsaturated fatty acids, fractionated triglycerides derived from coconut oil, fractionated liquid triglycerides derived from short chain 10 to 15 carbon atoms fatty acids, acetylated monoglycerides, acetylated diglycerides, acetylated triglycerides, olein known also as glyceral trioleate, palmitin known as glyceryl tripalmitate, stearin known also as glyceryl tristearate, lauric acid hexylester, oleic acid oleylester, glycolyzed ethoxylated glycerides of natural oils, branched fatty acids with 13 molecules of ethyleneoxide, and oleic acid decylester. The concentration of oil, or oil derivative in the emulsion formulation can be from about 1 wt % to about 40 wt %, with the wt % of all constituents in the emulsion preparation equal to 100 wt %. The oils are disclosed in Pharmaceutical Sciences by Remington, 17th Ed., pp. 403-405, (1985) published by Mark Publishing Co., in Encyclopedia of Chemistry, by Van Nostrand Reinhold, 4th Ed., pp. 644-645, (1984) published by Van Nostrand Reinhold Co.; and in U.S. Pat. No. 4,259,323, each of which is incorporated herein be reference in its entirety and for all purposes.

The amount of substances comprising levodopa and/or substances comprising carbidopa incorporated in the dosage forms of the present invention is generally from about 10% to about 90% by weight of the composition depending upon the therapeutic indication and the desired administration period, e.g., every 12 hours, every 24 hours, and the like. Depending on the dose of drug desired to be administered, one or more of the dosage forms can be administered.

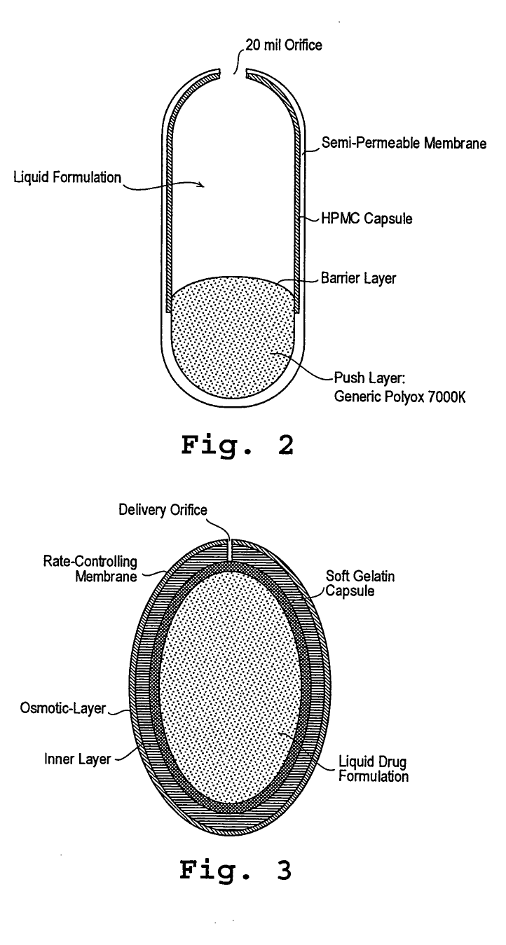

The osmotic dosage forms of the present invention can possess two distinct forms, a soft capsule form (shown in FIG. 3) and a hard capsule form (shown in FIG. 2). The soft capsule, as used by the present invention, preferably in its final form comprises one piece. The one-piece capsule is of a sealed construction encapsulating the drug formulation therein. The capsule can be made by various processes including the plate process, the rotary die process, the reciprocating die process, and the continuous process. An example of the plate process is as follows. The plate process uses a set of molds. A warm sheet of a prepared capsule lamina-forming material is laid over the lower mold and the formulation poured on it. A second sheet of the lamina-forming material is placed over the formulation followed by the top mold. The mold set is placed under a press and a pressure applied, with or without heat, to form a unit capsule. The capsules are washed with a solvent for removing excess agent formulation from the exterior of the capsule, and the air-dried capsule is encapsulated with a semipermeable wall. The rotary die process uses two continuous films of capsule lamina-forming material that are brought into convergence between a pair of revolving dies and an injector wedge. The process fills and seals the capsule in dual and coincident operations. In this process, the sheets of capsule lamina-forming material are fed over guide rolls, and then down between the wedge injector and the die rolls. The agent formulation to be encapsulated flows by gravity into a positive displacement pump. The pump meters the agent formulation through the wedge injector and into the sheets between the die rolls. The bottom of the wedge contains small orifices lined up with the die pockets of the die rolls. The capsule is about half-sealed when the pressure of pumped agent formulation forces the sheets into the die pockets, wherein the capsules are simultaneously filled, shaped, hermetically sealed and cut from the sheets of lamina-forming materials. The sealing of the capsule is achieved by mechanical pressure on the die rolls and by heating of the sheets of lamina-forming materials by the wedge. After manufacture, the agent formulation-filled capsules are dried in the presence of forced air, and a semipermeable lamina encapsulated thereto.

The reciprocating die process produces capsules by leading two films of capsule lamina-forming material between a set of vertical dies. The dies as they close, open, and close perform as a continuous vertical plate forming row after row of pockets across the film. The pockets are filled with agent formulation, and as the pockets move through the dies, they are sealed, shaped, and cut from the moving film as capsules filled with agent formulation. A semipermeable encapsulating lamina is coated thereon to yield the capsule. The continuous process is a manufacturing system that also uses rotary dies, with the added feature that the process can successfully fill active agent in dry powder form into a soft capsule, in addition to encapsulating liquids. The filled capsule of the continuous process is encapsulated with a semipermeable polymeric material to yield the capsule. Procedures for manufacturing soft capsules are disclosed in U.S. Pat. No. 4,627,850 and U.S. Pat. No. 6,419,952, each of which is hereby incorporated by reference in its entirety for all purposes.

The dosage forms of the present invention can also be made from an injection-moldable composition by an injection-molding technique. Injection-moldable compositions provided for injection-molding into the semipermeable wall comprise a thermoplastic polymer, or the compositions comprise a mixture of thermoplastic polymers and optional injection-molding ingredients. The thermoplastic polymer that can be used for the present purpose comprise polymers that have a low softening point, for example, below 200° C., preferably within the range of 40° C. to 180° C. The polymers, are preferably synthetic resins, addition polymerized resins, such as polyamides, resins obtained from diepoxides and primary alkanolamines, resins of glycerine and phthalic anhydrides, polymethane, polyvinyl resins, polymer resins with end-positions free or esterified carboxyl or caboxamide groups, for example with acrylic acid, acrylic amide, or acrylic acid esters, polycaprolactone, and its copolymers with dilactide, diglycolide, valerolactone and decalactone, a resin composition comprising polycaprolactone and polyalkylene oxide, and a resin composition comprising polycaprolactone, a polyalkylene oxide such as polyethylene oxide, poly(cellulose) such as poly(hydroxypropylmethylcellulose), poly(hydroxyethylmethylcellulose), and poly(hydroxypropylcellulose). The membrane forming composition can comprise optional membrane-forming ingredients such as polyethylene glycol, talcum, polyvinylalcohol, lactose, or polyvinyl pyrrolidone. The compositions for forming an injection-molding polymer composition can comprise 100% thermoplastic polymer. The composition in another embodiment comprises 10% to 99% of a thermoplastic polymer and 1% to 90% of a different polymer with the total equal to 100%. The invention provides also a thermoplastic polymer composition comprising 1% to 98% of a first thermoplastic polymer, 1% to 90% of a different, second polymer and 1% to 90% of a different, third polymer with all polymers equal to 100%. Representation composition comprises 20% to 90% of thermoplastic polycaprolactone and 10% to 80% of poly(alkylene oxide); a composition comprising 20% to 90% polycaprolactone and 10% to 60% of poly(ethylene oxide) with the ingredients equal to 100%; a composition comprising 10% to 97% of polycaprolactone, 10% to 97% poly(alkylene oxide), and 1% to 97% of poly(ethylene glycol) with all ingredients equal to 100%; a composition comprising 20% to 90% polycaprolactone and 10% to 80% of poly(hydroxypropylcellulose) with all ingredients equal to 100%; and a composition comprising 1% to 90% polycaprolactone, 1% to 90% poly(ethylene oxide), 1% to 90% poly(hydroxypropylcellulose) and 1% to 90% poly(ethylene glycol) with all ingredients equal to 100%. The percent, expressed is weight percent wt %.

In another embodiment of the invention, a composition for injection-molding to provide a membrane can be prepared by blending a composition comprising a polycaprolactone 63 wt %, polyethylene oxide 27 wt %, and polyethylene glycol 10 wt % in a conventional mixing machine, such as a Moriyama™ Mixer at 65° C. to 95° C., with the ingredients added to the mixer in the following addition sequence, polycaprolactone, polyethylene oxide and polyethylene glycol. In one example, all the ingredients are mixed for 135 minutes at a rotor speed of 10 to 20 rpm. Next, the blend is fed to a Baker Perkins Kneader™ extruder at 80° C. to 90° C., at a pump speed of 10 rpm and a screw speed of 22 rpm, and then cooled to 10° C. to 12° C., to reach a uniform temperature. Then, the cooled extruded composition is fed to an Albe Pelletizer, converted into pellets at 250° C., and a length of 5 mm. The pellets next are fed into an injection-molding machine, an Arburg Allrounder™ at 200° F. to 350° C. (93° C. to 177° C.), heated to a molten polymeric composition, and the liquid polymer composition forced into a mold cavity at high pressure and speed until the mold is filled and the composition comprising the polymers are solidified into a preselected shape. The parameters for the injection-molding consists of a band temperature through zone 1 to zone 5 of the barrel of 195° F. (91° C.) to 375° F., (191° C.), an injection-molding pressure of 1818 bar, a speed of 55 cm3 /s, and a mold temperature of 75° C. The injection-molding compositions and injection-molding procedures are disclosed in U.S. Pat. No. 5,614,578, herein incorporated by reference in its entirety and for all purposes.

Alternatively, the capsule can be made conveniently in two parts, with one part (the “cap”) slipping over and capping the other part (the “body”) as long as the capsule is deformable under the forces exerted by the expandable layer and seals to prevent leakage of the liquid, active agent formulation from between the telescoping portions of the body and cap. The two parts completely surround and capsulate the internal lumen that contains the liquid, active agent formulation, which can contain useful additives. The two parts can be fitted together after the body is filled with a preselected formulation. The assembly can be done by slipping or telescoping the cap section over the body section, and sealing the cap and body, thereby completely surrounding and encapsulating the formulation of active agent.

Soft capsules typically have a wall thickness that is greater than the wall thickness of hard capsules. For example, soft capsules can, for example, have a wall thickness on the order of 10-40 mils, about 20 mils being typical, whereas hard capsules can, for example, have a wall thickness on the order of 2-6 mils, about 4 mils being typical.

In one embodiment of the dosage system, a soft capsule can be of single unit construction and can be surrounded by an unsymmetrical hydro-activated layer as the expandable layer. The expandable layer will generally be unsymmetrical and have a thicker portion remote from the exit orifice. As the hydro-activated layer imbibes and/or absorbs external fluid, it expands and applies a push pressure against the wall of capsule and optional barrier layer and forces active agent formulation through the exit orifice. The presence of an unsymmetrical layer functions to assure that the maximum dose of agent is delivered from the dosage form, as the thicker section of layer distant from passageway swells and moves towards the orifice.

In yet another configuration, the expandable layer can be formed in discrete sections that do not entirely encompass an optionally barrier layer-coated capsule. The expandable layer can be a single element that is formed to fit the shape of the capsule at the area of contact. The expandable layer can be fabricated conveniently by tableting to form the concave surface that is complementary to the external surface of the barrier-coated capsule. Appropriate tooling such as a convex punch in a conventional tableting press can provide the necessary complementary shape for the expandable layer. In this case, the expandable layer is granulated and compressed, rather than formed as a coating. The methods of formation of an expandable layer by tableting are well known, having been described, for example in U.S. Pat. Nos. 4,915,949; 5,126,142; 5,660,861; 5,633,011; 5,190,765; 5,252,338; 5,620,705; 4,931,285; 5,006,346; 5,024,842; and 5,160,743, each of which is hereby incorporated by reference in its entirety for all purposes.

In some embodiments, a barrier layer can be first coated onto the capsule and then the tableted, expandable layer is attached to the barrier-coated capsule with a biologically compatible adhesive. Suitable adhesives include, for example, starch paste, aqueous gelatin solution, aqueous gelatin/glycerin solution, acrylate-vinylacetate based adhesives such as Duro-Tak adhesives (National Starch and Chemical Company), aqueous solutions of water soluble hydrophilic polymers such as hydroxypropyl methyl cellulose, hydroxymethyl cellulose, hydroxyethyl cellulose, and the like. That intermediate dosage form can be then coated with a semipermeable layer. The exit orifice is formed in the side or end of the capsule opposite the expandable layer section. As the expandable layer imbibes fluid, it will swell. Since it is constrained by the semipermeable layer, as it expands it will compress the barrier-coated capsule and express the liquid, active agent formulation from the interior of the capsule into the environment of use.

The hard capsules are typically composed of two parts, a cap and a body, which are fitted together after the larger body is filled with a preselected appropriate formulation. This can be done by slipping or telescoping the cap section over the body section, thus completely surrounding and encapsulating the useful agent formulation. Hard capsules can be made, for example, by dipping stainless steel molds into a bath containing a solution of a capsule lamina-forming material to coat the mold with the material. Then, the molds are withdrawn, cooled, and dried in a current of air. The capsule is stripped from the mold and trimmed to yield a lamina member with an internal lumen. The engaging cap that telescopically caps the formulation receiving body is made in a similar manner. Then, the closed and filled capsule can be encapsulated with a semipermeable lamina. The semipermeable lamina can be applied to capsule parts before or after parts and are joined into the final capsule. In another embodiment, the hard capsules can be made with each part having matched locking rings near their opened end that permit joining and locking together the overlapping cap and body after filling with formulation. In this embodiment, a pair of matched locking rings are formed into the cap portion and the body portion, and these rings provide the locking means for securely holding together the capsule. The capsule can be manually filled with the formulation, or they can be machine filled with the formulation. In the final manufacture, the hard capsule is encapsulated with a semipermeable lamina permeable to the passage of fluid and substantially impermeable to the passage of useful agent. Methods of forming hard cap dosage forms are described in U.S. Pat. No. 6,174,547, U.S. Pat. Nos. 6,596,314, 6,419,952, and 6,174,547, each of which is incorporated herein by reference in its entirety and for all purposes.

The hard and soft capsules can comprise, for example, gelatin; gelatin having a viscosity of 15 to 30 millipoises and a bloom strength up to 150 grams; gelatin having a bloom value of 160 to 250; a composition comprising gelatin, glycerine, water and titanium dioxide; a composition comprising gelatin, erythrosin, iron oxide and titanium dioxide; a composition comprising gelatin, glycerine, sorbitol, potassium sorbate and titanium dioxide; a composition comprising gelatin, acacia glycerine, and water; and the like. Materials useful for forming capsule wall are known in U.S. Pat. Nos. 4,627,850; and in 4,663,148, each of which is hereby incorporated by reference in its entirety for all purposes. Alternatively, the capsules can be made out of materials other than gelatin (see for example, products made by BioProgres plc).

The capsules typically can be provided, for example, in sizes from about 3 to about 22 minims (1 minimim being equal to 0.0616 ml) and in shapes of oval, oblong or others. They can be provided in standard shape and various standard sizes, conventionally designated as (000), (00), (0), (1), (2), (3), (4), and (5). The largest number corresponds to the smallest size. Non-standard shapes can be used as well. In either case of soft capsule or hard capsule, non-conventional shapes and sizes can be provided if required for a particular application.

The osmotic devices of the present invention comprise a semipermeable wall permeable to the passage of exterior biological fluid and substantially impermeable to the passage of drug formulation. The selectively permeable composition used for forming the wall are essentially non-erodible and they are insoluble in biological fluids during the life of the osmotic system. The semipermeable wall comprises a composition that does not adversely affect the host, the drug formulation, an osmopolymer, osmagent and the like. Representative polymers for forming semipermeable wall comprise semipermeable homopolymers, semipermeable copolymers, and the like. In one presently preferred embodiment, the compositions can comprise cellulose esters, cellulose ethers, and cellulose ester-ethers. The cellulosic polymers typically have a degree of substitution, “D.S.”, on their anhydroglucose unit from greater than 0 up to 3 inclusive. By degree of substitution is meant the average number of hydroxyl groups originally present on the anhydroglucose unit that are replaced by a substituting group, or converted into another group. The anhydroglucose unit can be partially or completely substituted with groups such as acyl, alkanoyl, alkenoyl, aroyl, alkyl, alkoxy, halogen, carboalkyl, alkylcarbamate, alkylcarbonate, alkylsulfonate, alkylsulfamate, semipermeable polymer forming groups, and the like. The semipermeable compositions typically include a member selected from the group consisting of cellulose acylate, cellulose diacylate, cellulose triacylate, cellulose triacetate, cellulose acetate, cellulose diacetate, cellulose triacetate, mono-, di- and tri-cellulose alkanylates, mono-, di-, and tri-alkenylates, mono-, di-, and tri-aroylates, and the like. Exemplary polymers can include, for example, cellulose acetate have a D.S. of 1.8 to 2.3 and an acetyl content of 32 to 39.9%; cellulose diacetate having a D.S. of 1 to 2 and an acetyl content of 21 to 35%, cellulose triacetate having a D.S. of 2 to 3 and an acetyl content of 34 to 44.8%, and the like. More specific cellulosic polymers include cellulose propionate having a D.S. of 1.8 and a propionyl content of 38.5%; cellulose acetate propionate having an acetyl content of 1.5 to 7% and an acetyl content of 39 to 42%; cellulose acetate propionate having an acetyl content of 2.5 to 3%, an average propionyl content of 39.2 to 45%, and a hydroxyl content of 2.8 to 5.4%; cellulose acetate butyrate having a D.S. of 1.8, an acetyl content of 13 to 15%, and a butyryl content of 34 to 39%; cellulose acetate butyrate having an acetyl content of 2 to 29%, a butyryl content of 17 to 53%, and a hydroxyl content of 0.5 to 4.7%; cellulose triacylates having a D.S. of 2.6 to 3 such as cellulose trivalerate, cellulose trilamate, cellulose tripalmitate, cellulose trioctanoate, and cellulose tripropionate; cellulose diesters having a D.S. of 2.2 to 2.6 such as cellulose disuccinate, cellulose dipalmitate, cellulose dioctanoate, cellulose dicarpylate, and the like; mixed cellulose esters such as cellulose acetate valerate, cellulose acetate succinate, cellulose propionate succinate, cellulose acetate octanoate, cellulose valerate palmitate, cellulose acetate heptonate, and the like. Semipermeable polymers are known in U.S. Pat. No. 4,077,407 and they can be synthesized by procedures described in Encyclopedia of Polymer Science and Technology, Vol. 3, pages 325 to 354, 1964, published by Interscience Publishers, Inc., New York; each of which is hereby incorporated by reference in its entirety for all purposes. Additional semipermeable polymers for forming the semipermeable wall can comprise, for example, cellulose acetaldehyde dimethyl acetate; cellulose acetate ethylcarbamate; cellulose acetate methylcarbamate; cellulose dimethylaminoacetate; semipermeable polyamide; semipermeable polyurethanes; semipermeable sulfonated polystyrenes; cross-linked selectively semipermeable polymers formed by the coprecipitation of a polyanion and a polycation as disclosed in U.S. Pat. Nos. 3,173,876; 3,276,586; 3,541,005; 3,541,006; and 3,546,142, each of which is hereby incorporated by reference in its entirety for all purposes; semipermeable polymers as disclosed in U.S. Pat. No. 3,133,132, hereby incorporated by reference in its entirety for all purposes; semipermeable polystyrene derivatives; semipermeable poly (sodium styrenesulfonate); semipermeable poly (vinylbenzyltremethylammonium chloride); semipermeable polymers, exhibiting a fluid permeability of 10-5 to 10-2 (cc. mil/cm hr.atm) expressed as per atmosphere of hydrostatic or osmotic pressure differences across a semipermeable wall. The polymers are known to the art in U.S. Pat. Nos. 3,845,770; 3,916,899; and 4,160,020; and in Handbook of Common Polymers, by Scott, J. R., and Roff, W. J., 1971, published by CRC Press, Cleveland. Ohio, each of which is hereby incorporated by reference in its entirety for all purposes.

The semipermeable wall can also comprise a flux regulating agent. The flux regulating agent is a compound added to assist in regulating the fluid permeability or flux through the wall. The flux regulating agent can be a flux enhancing agent or a decreasing agent. The agent can be preselected to increase or decrease the liquid flux. Agents that produce a marked increase in permeability to fluids such as water are often essentially hydrophilic, while those that produce a marked decrease to fluids such as water are essentially hydrophobic. The amount of regulator in the wall when incorporated therein generally is from about 0.01% to 20% by weight or more. The flux regulator agents in one embodiment that increase flux include, for example, polyhydric alcohols, polyalkylene glycols, polyalkylenediols, polyesters of alkylene glycols, and the like. Typical flux enhancers include polyethylene glycol 300, 400, 600, 1500, 4000, 6000, poly(ethylene glycol-co-propylene glycol), and the like; low molecular weight gylcols such as polypropylene glycol, polybutylene glycol and polyamylene glycol: the polyalkylenediols such as poly(1,3-propanediol), poly(1,4-butanediol), poly(1,6-hexanediol), and the like; aliphatic diols such as 1,3-butylene glycol, 1,4-pentamethylene glycol, 1,4-hexamethylene glycol, and the like; alkylene triols such as glycerine, 1,2,3-butanetriol, 1,2,4-hexanetriol, 1,3,6-hexanetriol and the like; esters such as ethylene glycol dipropionate, ethylene glycol butyrate, butylene glucol dipropionate, glycerol acetate esters, and the like. Representative flux decreasing agents include, for example, phthalates substituted with an alkyl or alkoxy or with both an alkyl and alkoxy group such as diethyl phthalate, dimethoxyethyl phthalate, dimethyl phthalate, and [di(2-ethylhexyl)phthalate], aryl phthalates such as triphenyl phthalate, and butyl benzyl phthalate; insoluble salts such as calcium sulphate, barium sulphate, calcium phosphate, and the like; insoluble oxides such as titanium oxide; polymers in powder, granule and like form such as polystyrene, polymethylmethacrylate, polycarbonate, and polysulfone; esters such as citric acid esters esterfied with long chain alkyl groups; inert and substantially water impermeable fillers; resins compatible with cellulose based wall forming materials, and the like.

Other materials that can be used to form the semipermeable wall for imparting flexibility and elongation properties to the wall, for making the wall less-to-nonbrittle and to render tear strength, include, for example, phthalate plasticizers such as dibenzyl phthalate, dihexyl phthalate, butyl octyl phthalate, straight chain phthalates of six to eleven carbons, di-isononyl phthalte, di-isodecyl phthalate, and the like. The plasticizers include nonphthalates such as triacetin, dioctyl azelate, epoxidized tallate, tri-isoctyl trimellitate, tri-isononyl trimellitate, sucrose acetate isobutyrate, epoxidized soybean oil, and the like. The amount of plasticizer in a wall when incorporated therein is about 0.01% to 20% weight, or higher.

The semipermeable wall surrounds and forms a compartment containing a plurality of layers, one of which is an expandable layer which in some embodiments, can contain osmotic agents. The expandable layer comprises in one embodiment a hydroactivated composition that swells in the presence of water, such as that present in gastric fluids. Conveniently, it can comprise an osmotic composition comprising an osmotic solute that exhibits an osmotic pressure gradient across the semipermeable layer against an external fluid present in the environment of use. In another embodiment, the hydro-activated layer comprises a hydrogel that imbibes and/or absorbs fluid into the layer through the outer semipermeable wall. The semipermeable wall is non-toxic. It maintains its physical and chemical integrity during operation and it is essentially free of interaction with the expandable layer.

The expandable layer in one preferred embodiment comprises a hydroactive layer comprising a hydrophilic polymer, also known as osmopolymers. The osmopolymers exhibit fluid imbibition properties. The osmopolymers are swellable, hydrophilic polymers, which osmopolymers interact with water and biological aqueous fluids and swell or expand to an equilibrium state. The osmopolymers exhibit the ability to swell in water and biological fluids and retain a significant portion of the imbibed fluid within the polymer structure. The osmopolymers swell or expand to a very high degree, usually exhibiting a 2 to 50 fold volume increase. The osmopolymers can be noncross-linked or cross-linked. The swellable, hydrophilic polymers are in one embodiment lightly cross-linked, such cross-links being formed by covalent or ionic bonds or residue crystalline regions after swelling. The osmopolymers can be of plant, animal or synthetic origin.

The osmopolymers are hydrophilic polymers. Hydrophilic polymers suitable for the present purpose include poly (hydroxy-alkyl methacrylate) having a molecular weight of from 30,000 to 5,000,000; poly (vinylpyrrolidone) having a molecular weight of from 10,000 to 360,000; anionic and cationic hydrogels; polyelectrolytes complexes; poly (vinyl alcohol) having a low acetate residual, cross-linked with glyoxal, formaldehyde, or glutaraldehyde and having a degree of polymerization of from 200 to 30,000; a mixture of methyl cellulose, cross-linked agar and carboxymethyl cellulose; a mixture of hydroxypropyl methylcellulose and sodium carboxymethylcellulose; a mixture of hydroxypropyl ethylcellulose and sodium carboxymethyl cellulose, a mixture of sodium carboxymethylcellulose and methylcellulose, sodium carboxymethylcellulose; potassium carboxymethylcellulose; a water insoluble, water swellable copolymer formed from a dispersion of finely divided copolymer of maleic anhydride with styrene, ethylene, propylene, butylene or isobutylene crosslinked with from 0.001 to about 0.5 moles of saturated cross-linking agent per mole of maleic anhydride per copolymer; water swellable polymers of N-vinyl lactams; polyoxyethylene-polyoxypropylene gel; carob gum; polyacrylic gel; polyester gel; polyuria gel; polyether gel, polyamide gel; polycellulosic gel; polygum gel; initially dry hydrogels that imbibe and absorb water which penetrates the glassy hydrogel and lowers its glass temperature; and the like.

Representative of other osmopolymers can comprise polymers that form hydrogels such as Carbopol™. acidic carboxypolymer, a polymer of acrylic acid cross-linked with a polyallyl sucrose, also known as carboxypolymethylene, and carboxyvinyl polymer having a molecular weight of 250,000 to 4,000,000; Cyanamer™ polyacrylamides; cross-linked water swellable indenemaleic anhydride polymers; Good-rite™ polyacrylic acid having a molecular weight of 80,000 to 200,000; Polyox™ polyethylene oxide polymer having a molecular weight of 100,000 to 5,000,000 and higher; starch graft copolymers; Aqua-Keeps™ acrylate polymer polysaccharides composed of condensed glucose units such as diester cross-linked polygluran; and the like. Representative polymers that form hydrogels are known to the prior art in U.S. Pat. No. 3,865,108; U.S. Pat. No. 4,002,173; U.S. Pat. No. 4,207,893; and in Handbook of Common Polymers, by Scott and Roff, published by the Chemical Rubber Co., Cleveland, Ohio, each of which is hereby incorporated by reference in its entirety for all purposes. The amount of osmopolymer comprising a hydro-activated layer can be from about 5% to 100%.

The expandable layer in another manufacture can comprise an osmotically effective compound that comprises inorganic and organic compounds that exhibit an osmotic pressure gradient across a semipermeable wall against an external fluid. The osmotically effective compounds, as with the osmopolymers, imbibe fluid into the osmotic system, thereby making available fluid to push against the inner wall, i.e., in some embodiments, the barrier layer and/or the wall of the soft or hard capsule for pushing active agent from the dosage form. The osmotically effective compounds are known also as osmotically effective solutes, and also as osmagents. Osmotically effective solutes that can be used comprise magnesium sulfate, magnesium chloride, potassium sulfate, sodium sulfate, lithium sulfate, potassium acid phosphate, mannitol, urea, inositol, magnesium succinate, tartaric acid, carbohydrates such as raffinose, sucrose, glucose, lactose, sorbitol, and mixtures therefor. The amount of osmagent in can be from about 5% to 100% of the weight of the layer. The expandable layer optionally comprises an osmopolymer and an osmagent with the total amount of osmopolymer and osmagent equal to 100%. Osmotically effective solutes are known to the prior art as described in U.S. Pat. No. 4,783,337, incorporated herein by reference in its entirety for all purposes.

In certain embodiments, the dosage forms further can comprise a barrier layer. The barrier layer in certain embodiments is deformable under the pressure exerted by the expandable layer and will be impermeable (or less permeable) to fluids and materials that can be present in the expandable layer, the liquid active agent formulation and in the environment of use, during delivery of the active agent formulation. A certain degree of permeability of the barrier layer can be permitted if the delivery rate of the active agent formulation is not detrimentally effected. However, it is preferred that barrier layer not completely transport through it fluids and materials in the dosage form and the environment of use during the period of delivery of the active agent. The barrier layer can be deformable under forces applied by expandable layer so as to permit compression of capsule to force the liquid, active agent formulation from the exit orifice. In some embodiments, the barrier layer will be deformable to such an extent that it create a seal between the expandable layer and the semipermeable layer in the area where the exit orifice is formed. In that manner, the barrier layer will deform or flow to a limited extent to seal the initially, exposed areas of the expandable layer and the semipermeable layer when the exit orifice is being formed, such as by drilling or the like, or during the initial stages of operation. When sealed, the only avenue for liquid permeation into the expandable layer is through the semipermeable layer, and there is no back-flow of fluid into the expandable layer through the exit orifice.

Suitable materials for forming the barrier layer can include, for example, polyethylene, polystyrene, ethylene-vinyl acetate copolymers, polycaprolactone and Hytrel™ polyester elastomers (Du Pont), cellulose acetate, cellulose acetate pseudolatex (such as described in U.S. Pat. No. 5,024,842), cellulose acetate propionate, cellulose acetate butyrate, ethyl cellulose, ethyl cellulose pseudolatex (such as Surelease™ as supplied by 10 Colorcon, West Point, Pa. or Aquacoat™ as supplied by FMC Corporation, Philadelphia, Pa.), nitrocellulose, polylactic acid, poly-glycolic acid, polylactide glycolide copolymers, collagen, polyvinyl alcohol, polyvinyl acetate, polyethylene vinylacetate, polyethylene teraphthalate, polybutadiene styrene, polyisobutylene, polyisobutylene isoprene copolymer, polyvinyl chloride, polyvinylidene chloride-vinyl chloride copolymer, copolymers of acrylic acid and methacrylic acid esters, copolymers of methylmethacrylate and ethylacrylate, latex of acrylate esters (such as Eudragit™ supplied by RohmPharma, Darmstaat, Germany), polypropylene, copolymers of propylene oxide and ethylene oxide, propylene oxide ethylene oxide block copolymers, ethylenevinyl alcohol copolymer, polysulfone, ethylene vinylalcohol copolymer, polyxylylenes, polyalkoxysilanes, polydimethyl siloxane, polyethylene glycol-silicone elastomers, electromagnetic irradiation crosslinked acrylics, silicones, or polyesters, thermally crosslinked acrylics, silicones, or polyesters, butadiene-styrene rubber, and blends of the above.

Preferred materials can include cellulose acetate, copolymers of acrylic acid and methacrylic acid esters, copolymers of methylmethacrylate and ethylacrylate, and latex of acrylate esters. Preferred copolymers can include poly (butyl methacrylate), (2-dimethylaminoethyl)methacrylate, methyl methacrylate) 1:2:1, 150,000, sold under the trademark EUDRAGIT E; poly (ethyl acrylate, methyl methacrylate) 2:1, 800,000, sold under the trademark EUDRAGIT NE 30 D; poly (methacrylic acid, methyl methacrylate) 1: 1, 135,000, sold under the trademark EUDRAGIT L; poly (methacrylic acid, ethyl acrylate) 1: 1, 250,000, sold under the trademark EUDRAGIT L; poly (methacrylic acid, methyl methacrylate) 1:2, 135,000, sold under the trademark EUDRAGIT S; poly (ethyl acrylate, methyl methacrylate, trimethylammonioethyl methacrylate chloride) 1:2:0.2, 150,000, sold under the trademark EUDRAGIT RL; poly (ethyl acrylate, methyl methacrylate, trimethylammonioethyl methacrylate chloride) 1:2:0.1, 150,000, sold as EUDRAGIT RS. In each case, the ratio x:y:z indicates the molar proportions of the monomer units and the last number is the number average molecular weight of the polymer. Especially preferred are cellulose acetate containing plasticizers such as acetyl tributyl citrate and ethylacrylate methylmethylacrylate copolymers such as Eudragit NE.

The foregoing materials for use as the barrier layer can be formulated with plasticizers to make the barrier layer suitably deformable such that the force exerted by the expandable layer will collapse the compartment formed by the barrier layer to dispense the liquid, active agent formulation. Examples of typical plasticizers are as follows: polyhydric alcohols, triacetin, polyethylene glycol, glycerol, propylene glycol, acetate esters, glycerol triacetate, triethyl citrate, acetyl triethyl citrate, glycerides, acetylated monoglycerides, oils, mineral oil, castor oil and the like. The plasticizers can be blended into the material in amounts of 10-50 weight percent based on the weight of the material.

The various layers forming the barrier layer, expandable layer and semipermeable layer can be applied by conventional coating methods such as described in U.S. Pat. No. 5,324,280, incorporated herein by reference in its entirety for all purposes. While the barrier layer, expandable layer and semipermeable wall have been illustrated and described for convenience as single layers, each of those layers can be composites of several layers. For example, for particular applications it may be desirable to coat the capsule with a first layer of material that facilitates coating of a second layer having the permeability characteristics of the barrier layer. In that instance, the first and second layers comprise the barrier layer. Similar considerations would apply to the semipermeable layer and the expandable layer.

The exit orifice can be formed by mechanical drilling, laser drilling, eroding an erodible element, extracting, dissolving, bursting, or leaching a passageway former from the composite wall. The exit orifice can be a pore formed by leaching sorbitol, lactose or the like from a wall or layer as disclosed in U.S. Pat. No. 4,200,098, herein incorporated by reference in its entirety for all purposes. This patent discloses pores of controlled-size porosity formed by dissolving, extracting, or leaching a material from a wall, such as sorbitol from cellulose acetate. A preferred form of laser drilling is the use of a pulsed laser that incrementally removes material from the composite wall to the desired depth to form the exit orifice.

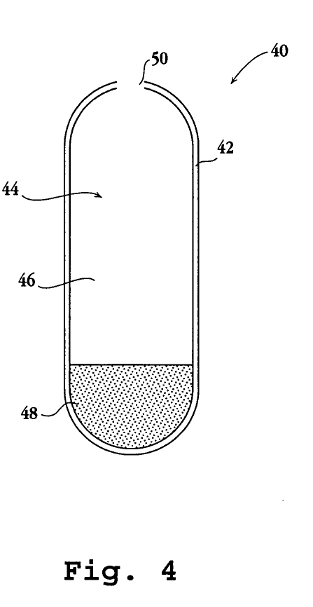

FIG. 4 is a schematic illustration of another exemplary osmotic dosage form. Dosage forms of this type are described in detail in U.S. Pat. Nos. 4,612,008; 5,082,668; and 5,091,190, which are incorporated by reference herein. In brief, dosage form 40, shown in cross-section, has a semi-permeable wall 42 defining an internal compartment 44. Internal compartment 44 contains a bilayered-compressed core having a drug layer 46 and a push layer 48. As will be described below, push layer 48 is a displacement composition that is positioned within the dosage form such that as the push layer expands during use, the materials forming the drug layer are expelled from the dosage form via one or more exit ports, such as exit port 50. The push layer can be positioned in contacting layered arrangement with the drug layer, as illustrated in FIG. 4, or can have one or more intervening layers separating the push layer and drug layer.

Drug layer 46 comprises substances comprising levodopa and/or substances comprising carbidopa in an admixture with selected excipients, such as those discussed above with reference to FIG. 3. An exemplary dosage form can have a drug layer comprised of a complex, a poly(ethylene oxide) as a carrier, sodium chloride as an osmagent, hydroxypropylmethylcellulose as a binder, and magnesium stearate as a lubricant.

Push layer 48 comprises osmotically active component(s), such as one or more polymers that imbibes an aqueous or biological fluid and swells, referred to in the art as an osmopolymer. Osmopolymers are swellable, hydrophilic polymers that interact with water and aqueous biological fluids and swell or expand to a high degree, typically exhibiting a 2-50 fold volume increase. The osmopolymer can be non-crosslinked or crosslinked, and in a preferred embodiment the osmopolymer is at least lightly crosslinked to create a polymer network that is too large and entangled to easily exit the dosage form during use. Examples of polymers that may be used as osmopolymers are provided in the references noted above that describe osmotic dosage forms in detail. A typical osmopolymer is a poly(alkylene oxide), such as poly(ethylene oxide), and a poly(alkali carboxymethylcellulose), where the alkali is sodium, potassium, or lithium. Additional excipients such as a binder, a lubricant, an antioxidant, and a colorant may also be included in the push layer. In use, as fluid is imbibed across the semi-permeable wall, the osmopolymer(s) swell and push against the drug layer to cause release of the drug from the dosage form via the exit port(s).

The push layer can also include a component referred to as a binder, which is typically a cellulose or vinyl polymer, such as poly-n-vinylamide, poly-n-vinylacetamide, poly(vinyl pyrrolidone), poly-n-vinylcaprolactone, poly-n-vinyl-5-methyl-2-pyrrolidone, and the like. The push layer can also include a lubricant, such as sodium stearate or magnesium stearate, and an antioxidant to inhibit the oxidation of ingredients. Representative antioxidants include, but are not limited to, ascorbic acid, ascorbyl palmitate, butylated hydroxyanisole, a mixture of 2 and 3 tertiary-butyl-4-hydroxyanisole, and butylated hydroxytoluene.

An osmagent may also be incorporated into the drug layer and/or the push layer of the osmotic dosage form. Presence of the osmagent establishes an osmotic activity gradient across the semi-permeable wall. Exemplary osmagents include salts, such as sodium chloride, potassium chloride, lithium chloride, etc. and sugars, such as raffinose, sucrose, glucose, lactose, and carbohydrates.

With continuing reference to FIG. 4, the dosage form can optionally include an overcoat (not shown) for color coding the dosage forms according to dose or for providing an immediate release of substances comprising levodopa and/or substances comprising carbidopa or other drugs.

In use, water flows across the wall and into the push layer and the drug layer. The push layer imbibes fluid and begins to swell and, consequently, pushes on drug layer 44 causing the material in the layer to be expelled through the exit orifice and into the gastrointestinal tract. Push layer 48 is designed to imbibe fluid and continue swelling, thus continually expelling substances comprising levodopa and/or substances comprising carbidopa from the drug layer throughout the period during which the dosage form is in the gastrointestinal tract. In this way, the dosage form provides a supply of substances comprising levodopa and/or substances comprising carbidopa to the gastrointestinal tract for a specified window.

In an embodiment, inventive dosage forms comprise two or more forms of substances comprising levodopa and/or substances comprising carbidopa so that a first form of substances comprising levodopa and/or substances comprising carbidopa is available for absorption in the upper G.I. tract and a second form is presented for absorption in the lower G.I. tract. This can facilitate optimal absorption in circumstances wherein different characteristics are needed to optimize absorption throughout the G.I. tract. Such an embodiment is achievable using a tri-layered osmotic dosage form

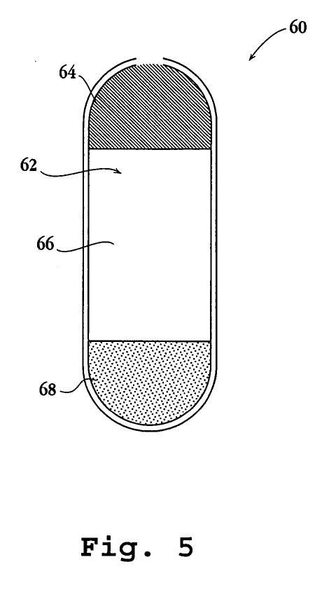

A specific exemplary dosage form comprising a first and second form of substances comprising levodopa is shown in FIG. 5. Osmotic dosage form 60 has a tri-layered core 62 comprised of a first layer 64 of a first form of a substance comprising levodopa, a second layer 66 comprising a second form of a substance comprising levodopa, and a third layer 68 referred to as a push layer. A tri-layered dosage form is prepared to have a first layer of 85.0 wt % of first form of a substance comprising levodopa, 10.0 wt % polyethylene oxide of 100,000 molecular weight, 4.5 wt % polyvinylpyrrolidone having a molecular weight of about 35,000 to 40,000, and 0.5 wt % magnesium stearate. The second layer is comprised 93.0 wt % of a second form of a substance comprising levodopa, 5.0 wt % polyethylene oxide 5,000,000 molecular weight, 1.0 wt % polyvinylpyrrolidone having molecular weight of about 35,000 to 40,000, and 1.0 wt % magnesium stearate.

The push layer consists of 63.67 wt % of polyethylene oxide, 30.00 wt % sodium chloride, 1.00 wt % ferric oxide, 5.00 wt % hydroxypropylmethylcellulose, 0.08 wt % butylated hydroxytoluene and 0.25 wt % magnesium stearate. The semi-permeable wall is comprised of 80.0 wt % cellulose acetate having a 39.8 % acetyl content and 20.0 % wt polyoxyethylene-polyoxypropylene copolymer.

Dissolution rates of dosage forms, such as those shown in FIGS. 2-5, can be determined according to a general procedure such as that set forth in Example 6. In general, release of drug formulation from the dosage form begins after contact with an aqueous environment. In the dosage form illustrated in FIG. 2, the drug moiety-transport moiety complex, present in the layer adjacent the exit orifice, is released after contact with an aqueous environment and continues for the lifetime of the device. The dosage form illustrated in FIG. 5 provides an initial release of drug moiety salt, present in the drug layer adjacent the exit orifice, with release of drug moiety-transport moiety complex occurring subsequently. It will be appreciated that this dosage form is designed to release drug moiety salt while in transit in the upper G.I. tract, corresponding approximately to the first eight hours of transit. The complex is released as the dosage form travels through the lower G.I. tract, approximately corresponding to times longer than about 8 hours after ingestion. This design takes advantage of the increased lower G.I. tract absorption provided by the complex.

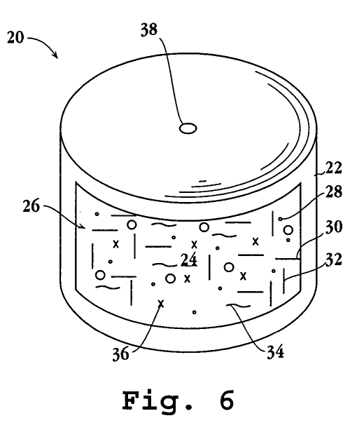

An exemplary dosage form, referred to in the art as an elementary osmotic pump dosage form, is shown in FIG. 6. Dosage form 20, shown in a cutaway view, is also referred to as an elementary osmotic pump, and is comprised of a semi-permeable wall 22 that surrounds and encloses an internal compartment 24. The internal compartment contains a single component layer referred to herein as a drug layer 26, comprising substances comprising levodopa and/or substances comprising carbidopa 28 in an admixture with selected excipients. The excipients are adapted to provide an osmotic activity gradient for attracting fluid from an external environment through wall 22 and for forming deliverable substances comprising levodopa and/or substances comprising carbidopa formulation upon imbibition of fluid. The excipients may include a suitable suspending agent, also referred to herein as drug carrier 30, a binder 32, a lubricant 34, and an osmotically active agent referred to as an osmagent 36. Exemplary materials for each of these components are provided below.

Semi-permeable wall 22 of the osmotic dosage form is permeable to the passage of an external fluid, such as water and biological fluids, but is substantially impermeable to the passage of components in the internal compartment. Materials useful for forming the wall are essentially nonerodible and are substantially insoluble in biological fluids during the life of the dosage form. Representative polymers for forming the semi-permeable wall include homopolymers and copolymers, such as, cellulose esters, cellulose ethers, and cellulose ester-ethers. Flux-regulating agents can be admixed with the wall-forming material to modulate the fluid permeability of the wall. For example, agents that produce a marked increase in permeability to fluid such as water are often essentially hydrophilic, while those that produce a marked permeability decrease to water are essentially hydrophobic. Exemplary flux regulating agents include polyhydric alcohols, polyalkylene glycols, polyalkylenediols, polyesters of alkylene glycols, and the like.

In operation, the osmotic gradient across wall 22 due to the presence of osmotically-active agents causes gastric fluid to be imbibed through the wall, swelling of the drug layer, and formation of a deliverable formulation of substances comprising levodopa and/or substances comprising carbidopa (e.g., a solution, suspension, slurry or other flowable composition) within the internal compartment. The deliverable formulation is released through an exit 38 as fluid continues to enter the internal compartment. Even as 3ANBPA formulation is released from the dosage form, fluid continues to be drawn into the internal compartment, thereby driving continued release. In this manner, substances comprising levodopa and/or substances comprising carbidopa is released in a sustained manner over an extended time period.

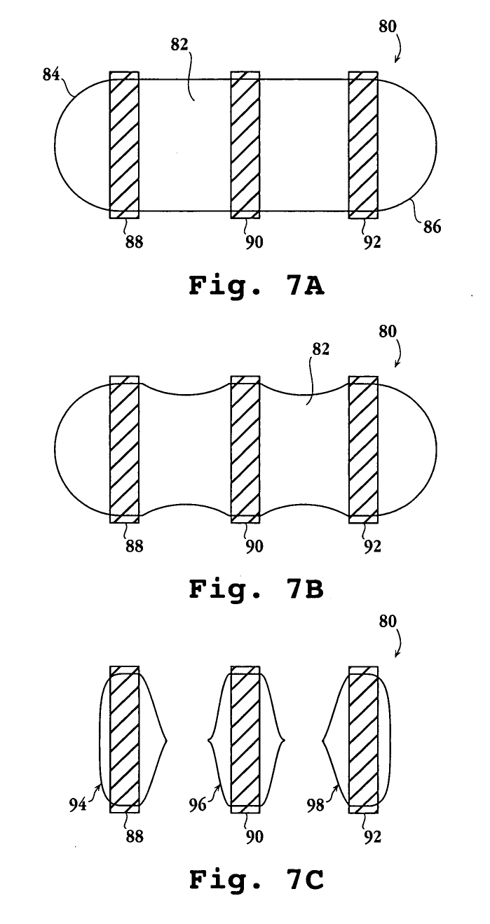

FIGS. 7A-7C illustrate another exemplary dosage form, known in the art and described in U.S. Pat. Nos. 5,534,263; 5,667,804; and 6,020,000, which are specifically incorporated by reference herein. Briefly, a cross-sectional view of a dosage form 80 is shown prior to ingestion into the gastrointestinal tract in FIG. 7A. The dosage form is comprised of a cylindrically shaped matrix 82 comprising substances comprising levodopa and/or substances comprising carbidopa. Ends 84, 86 of matrix 82 are preferably rounded and convex in shape in order to ensure ease of ingestion. Bands 88, 90, and 92 concentrically surround the cylindrical matrix and are formed of a material that is relatively insoluble in an aqueous environment. Suitable materials are set forth in the patents noted above and in Example 6 below.

After ingestion of dosage form 80, regions of matrix 82 between bands 88, 90, 92 begin to erode, as illustrated in FIG. 7B. Erosion of the matrix initiates release of substances comprising levodopa and/or substances comprising carbidopa into the fluidic environment of the G.I. tract. As the dosage form continues transit through the G.I. tract, the matrix continues to erode, as illustrated in FIG. 7C. Here, erosion of the matrix has progressed to such an extent that the dosage form breaks into three pieces, 94, 96, 98. Erosion will continue until the matrix portions of each of the pieces have completely eroded. Bands 94, 96, 98 will thereafter be expelled from the G.I. tract.

It will be appreciated the dosage forms described in FIGS. 2-7 are merely exemplary of a variety of dosage forms designed for and capable of achieving delivery of the inventive moiety complex to the G.I. tract. Those of skill in the pharmaceutical arts can identify other dosage forms that would be suitable.

It should be noted that, in some cases, a non-controlled release dosage form may be desirable. For instance, levodopa and/or a levodopa complex and/or carbidopa and/or a carbidopa complex may be dosed using an immediate release dosage form if controlled delivery to the lower G.I. is not necessary for a specific clinical situation. This may be the situation, for example, if a swift onset of action is clinically desirable. Due to enhanced bioavailability, the inventive complexes may be administered as an IR dosage form to achieve a higher plasma levodopa concentration than Sinemet at an equivalent dose. The apparent dose sparing could lead to a reduced levodopa-induced delay in gastric emptying as suggested by DRC Robertson et. al. “The influence of levodopa on gastric emptying in man.” Br J Clin Pharmacol 29:47-53 (1990). This may provide an IR dosage form of the inventive complexes with a more rapid onset of action than Sinemet®.

Typical doses of substances that comprise levodopa and/or substances that comprise carbidopa in the inventive dosage forms may vary broadly. The inventors note that the molecular weight of substances that comprise levodopa and/or substances that comprise carbidopa may vary significantly depending on whether it is administered as a loose ion-pair salt, a complex, a structural homolog, and so on. Therefore, the dosage strength of substances that comprise levodopa and/or substances that comprise carbidopa may need to be varied as the form incorporated into the dosage form is varied. The dose administered is generally adjusted in accord with the desired result for individual patients.

As the molecular weight is different for various forms of levodopa or carbidopa, it is confusing to report the dose for a form according to its weight equivalent. It is preferred to report them as the weight equivalent of levodopa or carbidopa monohydrate that is the form currently available on the market and known by most physicians (for example, the forms available in Sinemet™). For instance, the molecular weight of levodopa lauryl sulfate is 463.59, while the molecular weight of levodopa is 197.19. To dose 200 mg weight equivalent of levodopa, one would need to dose 470 mg of levodopa lauryl sulfate. On this basis, certain embodiments according to the invention may comprise a weight equivalent of form(s) of levodopa present in the dosage form ranging from about 10 mg to about 1000 mg, preferably from about 50 mg to about 900 mg, and more preferably from about 100 mg to about 400 mg. Particular dosage forms may contain about 10 mg, about 20 mg, about 30 mg, about 40 mg, about 50 mg, about 100 mg, about 150 mg, about 200 mg, about 300 mg, about 400 mg, about 500 mg, about 750 mg, or about 1000 mg weight equivalents in a given dosage form. Further, certain embodiments according to the invention may comprise a weight equivalent of form(s) of carbidopa present in the dosage form ranging from about 1 mg to about 300 mg, preferably from about 2.5 mg to about 250 mg, and more preferably from about 25 mg to about 100 mg. Particular dosage forms may contain about 1 mg, about 2 mg, about 2.5 mg, about 5 mg, about 10 mg, about 20 mg, about 40 mg, about 50 mg, about 60 mg, about 75 mg, about 100 mg, about 200 mg, or about 300 mg weight equivalents in a given dosage form. Preferred dosing regimens comprise twice daily (e.g. bid) or once per day (e.g. qd) dosing.

In an aspect, the invention provides a method for treating an indication, such as a disease or disorder, preferably a disease or disorder amenable to treatment by administration of levodopa, in a patient by administering a controlled delivery dosage form that comprises substances that comprise levodopa and/or substances that comprise carbidopa. In one embodiment, a composition comprising substances that comprise levodopa and/or substances that comprise carbidopa, and a pharmaceutically-acceptable vehicle, is administered to the patient via oral administration.

The present invention is further directed to a method of treatment comprising administering to a patient in need thereof, an oral controlled delivery dosage form comprising substances that comprise levodopa and/or substances that comprise carbidopa wherein the substances that comprise levodopa and/or substances that comprise carbidopa are released from the dosage form at a substantially zero order rate of release, preferably a zero order rate of release. A variety of controlled delivery dosage forms disclosed herein are capable of providing a substantially zero order rate of release, preferably a zero order rate of release. Such dosage forms comprise elementary osmotic pumps, matrix, and bi-layered osmotic dosage forms, as well as others known to one of skill in the art.

It is clinically desirable to maintain a reasonably consistent inhibition of peripheral decarboxylase through a reasonably consistent plasma carbidopa concentration. This leads to an reasonably constant plasma levodopa concentration that is desirable for maintaining the patients under control. K J Black et al. “Rapid intravenous loading of levodopa for human research: clinical results.” Journal of Neuroscience Methods 127:19-23 (2003); R Durso “Variable absorption of carbidopa affects both peripheral and central levodopa metabolism”. J Clin Pharmacol 40:854-60 (2000).

Limiting the periods wherein carbidopa or levodopa plasma drug concentrations are at least about fifteen percent of their respective Cmax may be useful to reduce potential side effects, and may provide dose sparing effects, that may be associated with continuously high (e.g. continuous infusion) plasma levels of levodopa. Continuously high levels of levodopa downregulates, and lower plasma levels may be able to restore, the sensitivity of dopamine receptors. This effect was reported for enteral infusion of levodopa in J. M. Cedarbaum et al., “Sustained enteral administration of levodopa increases and interrupted infusion decreases levodopa dose requirements.” Neurobiology 40:995-997 (June 1990), hereby incorporated by reference. The inventors hypothesize that this approach may be applied to the development of the inventive dosage forms to provide controlled delivery oral therapies possessing improved clinical results. One of skill can optimize the time during which carbidopa or levodopa plasma drug concentrations are not at least about fifteen percent of their respective Cmax to provide dosage forms having optimal performance for the patient.

In certain embodiments, the period during which the carbidopa plasma drug concentration is at least about fifteen percent of the Cmax, and the period during which levodopa plasma drug concentration is at least about fifteen percent of the Cmax may not be the same. For instance, the carbidopa plasma concentration may drop below about fifteen percent of the Cmax before the levodopa concentration drops below about fifteen percent of the Cmax, or vice versa.

In preferable embodiments, the controlled delivery dosing structure is adapted to controllably deliver substances that comprises levodopa and/or substances that comprises carbidopa contained by the controlled delivery dosing structure at an ascending rate of release effective to, after a single administration of the dosage form to a patient, provide a substantially zero order plasma profile of levodopa and/or carbidopa throughout a window of at least about ten hours duration.

These plasma profiles may be clinically advantageous because they provides for zero order delivery of substances comprising levodopa and/or substances comprising carbidopa during a period when immediate release levodopa and/or immediate release carbidopa would not provide such a zero order profile. Lack of a zero order profile may lead to the “on-off” problems and other problems noted above with regards to conventional dosage forms.

The ascending release rate embodiments are particularly useful in circumstances wherein the lower G.I. absorption is still less than the upper G.I. absorption. In such case, the ascending release rate can compensate in part for reduced lower G.I. absorption or even reduced absorption in areas of the upper G.I. that do not posses high levels of the active transporters that may be responsible for the primary transport of levodopa and carbidopa. In one ascending rate of release embodiment, the release rate over the first approximately 3 hours after dosing of an inventive dosage form is approximately 1/F fold that of the release rate beyond approximately 3 hours after dosing where
F=X/Y
and wherein X =bioavailability of levodopa or carbidopa when delivered to the lower G.I. as an inventive complex, and Y =bioavailability of levodopa or carbidopa when delivered to the upper G.I. as an inventive complex. Various ascending release rate profiles can be obtained by one of skill in the art by optimizing appropriate formulations. For instance, of skill in the art could adjust the dosage form shown in FIG. 5 to varying release rates so as to achieve a desired ascending release rate profile. Such adjustments are known to one of skill in the art.

In one embodiment, the inventive dosage forms may achieve an ascending release rate through the provision of more than one drug layer. In osmotic devices with multiple drug layers, a drug concentration gradient between the layers facilitates the achievement of an ascending drug release rate for an extended time period. For example, in one embodiment of the present invention, the osmotic dosage form comprises a first drug layer and a second drug layer, wherein the concentration of drug contained within the first layer is greater than the concentration of drug contained within the second layer, and the expandable layer is contained within a third layer. In outward order from the core of the dosage form is the expandable layer, the second drug layer, and the first drug layer. In operation through the cooperation of the dosage form components, substances comprising levodopa and/or substances comprising carbidopa are successively released, in a sustained and controlled manner, from the second drug layer and then from the first drug layer such that an ascending release rate over an extended time period is achieved.

The present invention is further directed to pharmaceutical compositions, as that term is defined herein, and to methods of administering pharmaceutical compositions to a patient in need thereof. Preferably the present invention is directed to methods of administering pharmaceutical compositions to a patient in need thereof in therapeutically effective amounts.

In an embodiment, the invention relates to a substance comprising: a complex comprising levodopa and a transport moiety, preferably wherein the transport moiety comprises an alkyl sulfate salt, more preferably wherein the alkyl sulfate salt comprises sodium lauryl sulfate. The invention also relates to a pharmaceutical composition comprising: the substance and a pharmaceutically-acceptable carrier, and to an oral dosage form comprising the pharmaceutical composition. In preferable embodiments, the oral dosage form further comprises carbidopa, or a carbidopa complex. In other preferable embodiments the oral dosage form comprises an oral controlled delivery dosage form, preferably the oral dosage form comprises an osmotic oral controlled delivery dosage form, more preferably the osmotic oral controlled delivery dosage form comprises a solid osmotic oral controlled delivery dosage form. In other preferable embodiments, the solid osmotic oral controlled delivery dosage form further comprises carbidopa, or the solid osmotic oral controlled delivery dosage form further comprises a carbidopa complex. In certain preferable embodiments, the osmotic oral controlled delivery dosage form comprises a liquid osmotic oral controlled delivery dosage form, which preferably comprises carbidopa or a carbidopa complex. The invention also relates to methods comprising administering any of the oral dosage forms above to a patient. The invention further relates to the oral dosage form wherein the controlled delivery dosage form controllably delivers the substance in a delivery dose pattern of from about 0 wt % to about 20 wt % in about 0 to about 4 hrs, about 20 wt % to about 50 wt % in about 0 to about 8 hrs, about 55 wt % to about 85 wt % in about 0 to about 14 hrs, and about 80 wt % to about 100 wt % in about 0 to about 24 hrs. The invention further relates to the oral dosage form wherein the controlled delivery dosage form controllably delivers the substance in a delivery dose pattern of from about 0 wt % to about 20 wt % in about 0 to about 4 hrs, about 20 wt % to about 50 wt % in about 0 to about 8 hrs, about 55 wt % to about 85 wt % in about 0 to about 14 hrs, and about 80 wt % to about 100 wt % in about 0 to about 20 hrs. The invention further relates to the oral dosage form wherein the controlled delivery dosage form controllably delivers the substance in a delivery dose pattern of from about 0 wt % to about 20 wt % in about 0 to about 2 hrs, about 20 wt % to about 50 wt % in about 0 to about 4 hrs, about 55 wt % to about 85 wt % in about 0 to about 7 hrs, and about 80 wt % to about 100 wt % in about 0 to about 8 hrs.

The invention relates to a method comprising: providing an alkyl sulfate salt; converting the alkyl sulfate salt to an acid form of the alkyl sulfate; contacting levodopa with the acid form of the alkyl sulfate to form a levodopa-alkyl sulfate complex; and isolating the complex. In other embodiments, the invention relates to the method wherein the alkyl sulfate salt comprises sodium lauryl sulfate. In other embodiments, the invention relates to the method wherein converting the alkyl sulfate salt to an acid form of the alkyl sulfate is performed using an ion exchange process.

The invention relates to a substance comprising: a complex comprising carbidopa and a transport moiety. In preferable embodiments of the substance, the transport moiety comprises an alkyl sulfate salt, preferably wherein the alkyl sulfate salt comprises sodium lauryl sulfate. In other embodiments, the invention relates to a pharmaceutical composition comprising the substance and a pharmaceutically-acceptable carrier. The invention also relates to an oral dosage form comprising the pharmaceutical composition, which preferably further comprises levodopa or a levodopa complex. In preferable embodiments, the oral dosage form comprises a controlled delivery oral dosage form, preferably the oral dosage form comprises an osmotic controlled delivery oral dosage form, more preferably the osmotic controlled delivery oral dosage form comprises a solid osmotic controlled delivery oral dosage form, still more preferably the solid osmotic controlled delivery oral dosage form further comprises levodopa or a levodopa complex. In other embodiments, the osmotic controlled delivery oral dosage form comprises a liquid osmotic controlled delivery oral dosage form, preferably wherein the liquid osmotic controlled delivery oral dosage form further comprises levodopa or a levodopa complex. The invention also relates to methods comprising administering the above oral dosage forms to a patient.

The invention further relates to the oral dosage forms, wherein the controlled delivery dosage form controllably delivers the substance in a delivery dose 25 pattern of from about 0 wt % to about 20 wt % in about 0 to about 4 hrs, about 20 wt % to about 50 wt % in about 0 to about 8 hrs, about 55 wt % to about 85 wt % in about 0 to about 14 hrs, and about 80 wt % to about 100 wt % in about 0 to about 24 hrs. In another embodiment, the controlled delivery dosage form controllably delivers the substance in a delivery dose pattern of from about 0 wt % to about 20 wt % in about 0 to 30 about 4 hrs, about 20 wt % to about 50 wt % in about 0 to about 8 hrs, about 55 wt % to about 85 wt % in about 0 to about 14 hrs, and about 80 wt % to about 100 wt % in about 0 to about 20 hrs. In yet another embodiment, the controlled delivery dosage form controllably delivers the substance in a delivery dose pattern of from about 0 wt % to about 20 wt % in about 0 to about 2 hrs, about 20 wt % to about 50 wt % in about 0 to about 4 hrs, about 55 wt % to about 85 wt % in about 0 to about 7 hrs, and about 80 wt % to about 100 wt % in about 0 to about 8 hrs.

The invention relates to a method comprising providing an alkyl sulfate salt; converting the alkyl sulfate salt to an acid form of the alkyl sulfate; contacting carbidopa with the acid form of the alkyl sulfate to form a levodopa-alkyl sulfate complex; and isolating the complex. In preferable embodiments, the alkyl sulfate salt comprises sodium lauryl sulfate. In preferable embodiments, converting the alkyl sulfate salt to an acid form of the alkyl sulfate is performed using an ion exchange process.

The invention relates to an oral dosage form comprising: (i) an oral controlled delivery dosing structure comprising structure that controllably delivers a substance that comprises levodopa and a substance that comprises carbidopa; wherein at least a portion of the substance that comprises levodopa and a portion substance that comprises carbidopa are contained by the controlled delivery dosing structure; and wherein the controlled delivery dosing structure is adapted to controllably deliver the portion of the substance that comprises levodopa and the portion of the substance that comprises carbidopa contained by the controlled delivery dosing structure at rates that are effective to, after a single administration of the dosage form to a patient:
a. provide a levodopa Cmax ranging from about 236 to about 988 ng/mL,
b. provide a levodopa AUC from about 3676 to about 15808 h·ng/mL, and
c. maintain a levodopa plasma drug concentration that is at least about fifteen percent of the levodopa Cmax throughout a window of at least about ten hours duration.
d. provide a carbidopa Cmax ranging from about 1 to about 500 ng/ml μmol/L,
e. provide an carbidopa AUC from about 20000 to about 200000 h·ng/mL, and
f. maintain a carbidopa plasma drug concentration that is at least about fifteen percent of the carbidopa Cmax throughout a window of at least about ten hours duration.

In preferable embodiments, the substance that comprises levodopa comprises: a levodopa complex or a levodopa prodrug. In other preferable embodiments, the substance that comprises carbidopa comprises: a carbidopa complex or a carbidopa prodrug.

In preferable embodiments of the dosage form, the controlled delivery dosing structure is adapted to controllably deliver the portion of the substance that comprises levodopa contained by the controlled delivery dosing structure at a rate that is effective to, after a single administration of the dosage form to a patient, maintain a levodopa plasma drug concentration that is at least about fifteen percent of the

levodopa Cmax throughout a window of at least about twelve hours duration. In other preferable embodiments of the dosage form, the controlled delivery dosing structure is adapted to controllably deliver the portion of the substance that comprises levodopa contained by the controlled delivery dosing structure at a rate that is effective to, after a single administration of the dosage form to a patient, maintain a levodopa plasma drug concentration that is at least about fifteen percent of the levodopa Cmax throughout a window of at least about sixteen hours duration. In preferable embodiments of the dosage form, the controlled delivery dosing structure is adapted to controllably deliver the portion of the substance that comprises levodopa contained by the controlled delivery dosing structure at a rate that is effective to, after a single administration of the dosage form to a patient, maintain a levodopa plasma drug concentration that is at least about fifteen percent of the levodopa Cmax throughout a window of at least about eighteen hours duration. In preferable embodiments of the dosage form, the controlled delivery dosing structure is adapted to controllably deliver the portion of the substance that comprises levodopa contained by the controlled delivery dosing structure at a rate that is effective to, after a single administration of the dosage form to a patient, maintain a levodopa plasma drug concentration that is at least about fifteen percent of the levodopa.Cmax throughout a window of at least about twenty hours duration. In preferable embodiments of the dosage form, the controlled delivery dosing structure is adapted to controllably deliver the portion of the substance that comprises carbidopa contained by the controlled delivery dosing structure at a rate that is effective to, after a single administration of the dosage form to a patient, maintain a carbidopa plasma drug concentration that is at least about fifteen percent of the carbidopa Cmax throughout a window of at least about twelve hours duration. In preferable embodiments of the dosage form, the controlled delivery dosing structure is adapted to controllably deliver the portion of the substance that comprises carbidopa contained by the controlled delivery dosing structure at a rate that is effective to, after a single administration of the dosage form to a patient, maintain a carbidopa plasma drug concentration that is at least about fifteen percent of the carbidopa Cmax throughout a window of at least about sixteen hours duration. In preferable embodiments of the dosage form, the controlled delivery dosing structure is adapted to controllably deliver the portion of the substance that comprises carbidopa contained by the controlled delivery dosing structure at a rate that is effective to, after a single administration of the dosage form to a patient, maintain a carbidopa plasma drug concentration that is at least about fifteen percent of the carbidopa Cmax throughout a window of at least about eighteen hours duration. In preferable embodiments of the dosage form, the controlled delivery dosing structure is adapted to controllably deliver the portion of the substance that comprises carbidopa contained by the controlled delivery dosing structure at a rate that is effective to, after a single administration of the dosage form to a patient, maintain a carbidopa plasma drug concentration that is at least about fifteen percent of the carbidopa Cmax throughout a window of at least about twenty hours duration.

The invention relates to an oral controlled delivery dosage form comprising an oral controlled delivery dosing structure comprising structure that controllably delivers a substance that comprises levodopa; wherein at least a portion of the substance that comprises levodopa is contained by the controlled delivery dosing structure; and wherein the controlled delivery dosing structure is adapted to controllably deliver the portion of the substance that comprises levodopa contained by the controlled delivery dosing structure at an ascending rate of release effective to, after a single administration of the dosage form to a patient, provide a substantially zero order levodopa plasma profile for a window of at least about six hours duration

In preferable embodiments of the oral controlled delivery dosage form, the controlled delivery dosing structure is adapted to controllably deliver the portion of the substance that comprises levodopa contained by the controlled delivery dosing structure at an ascending rate of release effective to, after a single administration of the dosage form to a patient, provide a substantially zero order levodopa plasma profile for a window of at least about twelve hours duration. In preferable embodiments of the oral controlled delivery dosage form, the controlled delivery dosing structure is adapted to controllably deliver the portion of the substance that comprises levodopa contained by the controlled delivery dosing structure at an ascending rate of release effective to, after a single administration of the dosage form to a patient, provide a substantially zero order levodopa plasma profile for a window of at least about sixteen hours duration. In preferable embodiments of the oral controlled delivery dosage form, the substance that comprises levodopa comprises: a levodopa complex or a levodopa prodrug.

Preferable embodiments of the oral controlled delivery dosage form further comprise: an oral controlled delivery dosing structure comprising structure that controllably delivers a substance that comprises carbidopa; wherein at least a portion of the substance that comprises carbidopa is contained by the controlled delivery dosing structure; and wherein the controlled delivery dosing structure is adapted to controllably deliver the portion of the substance that comprises carbidopa contained by the controlled delivery dosing structure at an ascending rate of release effective to, after a single administration of the dosage form to a patient, provide a substantially zero order carbidopa plasma profile for a window of at least about six hours duration.

In preferable embodiments of the oral controlled delivery dosage form, the controlled delivery dosing structure is adapted to controllably deliver the portion of the substance that comprises carbidopa contained by the controlled delivery dosing structure at an ascending rate of release effective to, after a single administration of the dosage form to a patient, provide a substantially zero order carbidopa plasma profile for a window of at least about twelve hours duration. In preferable embodiments of the oral controlled delivery dosage form, the controlled delivery dosing structure is adapted to controllably deliver the portion of the substance that comprises carbidopa contained by the controlled delivery dosing structure at an ascending rate of release effective to, after a single administration of the dosage form to a patient, provide a substantially zero order carbidopa plasma profile for a window of at least about sixteen hours duration

In preferable embodiments of the oral controlled delivery dosage form, the substance that comprises carbidopa comprises: a carbidopa complex or a carbidopa prodrug.

The invention relates to a composition comprising: levodopa; an alkyl sulfate salt; and a pharmaceutically-acceptable carrier. Preferably, in the composition, the alkyl sulfate salt comprises sodium lauryl sulfate. The invention also relates to an oral dosage form comprising the pharmaceutical composition, preferably wherein the oral dosage form further comprises carbidopa.

The invention relates to an oral dosage form comprising: (i) an oral controlled delivery dosing structure comprising structure that controllably delivers a substance that comprises levodopa; wherein at least a portion of the substance that comprises levodopa is contained by the controlled delivery dosing structure; and wherein the controlled delivery dosing structure is adapted to controllably deliver the portion of the substance that comprises levodopa contained by the controlled delivery dosing structure at rates that are effective to, after a single administration of the dosage form to a patient:
a. provide a levodopa Cmax ranging from about 236 to about 988 ng/mL,
b. provide a levodopa AUC from about 3676 to about 15808 h·ng/mL, and
c. maintain a levodopa plasma drug concentration that is at least about fifteen percent of the levodopa Cmax throughout a window of at least about ten hours duration.

In preferable embodiments, the substance that comprises levodopa comprises: a levodopa complex or a levodopa prodrug. In preferable embodiments, the controlled delivery dosing structure is adapted to controllably deliver the portion of the substance that comprises levodopa contained by the controlled delivery dosing structure at a rate that is effective to, after a single administration of the dosage form to a patient, maintain a levodopa plasma drug concentration that is at least about fifteen percent of the levodopa Cmax throughout a window of at least about twelve hours duration. In preferable embodiments, the controlled delivery dosing structure is adapted to controllably deliver the portion of the substance that comprises levodopa contained by the controlled delivery dosing structure at a rate that is effective to, after a single administration of the dosage form to a patient, maintain a levodopa plasma drug concentration that is at least about fifteen percent of the levodopa Cmax throughout a window of at least about sixteen hours duration. In preferable embodiments, the controlled delivery dosing structure is adapted to controllably deliver the portion of the substance that comprises levodopa contained by the controlled delivery dosing structure at a rate that is effective to, after a single administration of the dosage form to a patient, maintain a levodopa plasma drug concentration that is at least about fifteen percent of the levodopa Cmax throughout a window of at least about eighteen hours duration. In preferable embodiments, the controlled delivery dosing structure is adapted to controllably deliver the portion of the substance that comprises levodopa contained by the controlled delivery dosing structure at a rate that is effective to, after a single administration of the dosage form to a patient, maintain a levodopa plasma drug concentration that is at least about fifteen percent of the levodopa Cmax throughout a window of at least about twenty hours duration.

The invention relates to an oral dosage form comprising: (i) an oral controlled delivery dosing structure comprising structure that controllably delivers a substance that comprises carbidopa; wherein at least a portion of the substance that comprises carbidopa is contained by the controlled delivery dosing structure; and wherein the controlled delivery dosing structure is adapted to controllably deliver the portion of the substance that comprises carbidopa contained by the controlled delivery dosing structure at rates that are effective to, after a single administration of the dosage form to a patient:
a. provide a carbidopa Cmax ranging from about 1 to about 500 ng/ml μmol/L,
b. provide an carbidopa AUC from about 20000 to about 200000 h·ng/mL, and
c. maintain a carbidopa plasma drug concentration that is at least about fifteen percent of the carbidopa Cmax throughout a window of at least about ten hours duration.

In preferable embodiments, the substance that comprises carbidopa comprises: a carbidopa complex or a carbidopa prodrug. In preferable embodiments, the controlled delivery dosing structure is adapted to controllably deliver the portion of the substance that comprises carbidopa contained by the controlled delivery dosing structure at a rate that is effective to, after a single administration of the dosage form to a patient, maintain a carbidopa plasma drug concentration that is at least about fifteen percent of the carbidopa Cmax throughout a window of at least about twelve hours duration. In preferable embodiments, the controlled delivery dosing structure is adapted to controllably deliver the portion of the substance that comprises carbidopa contained by the controlled delivery dosing structure at a rate that is effective to, after a single administration of the dosage form to a patient, maintain a carbidopa plasma drug concentration that is at least about fifteen percent of the carbidopa Cmax throughout a window of at least about sixteen hours duration. In preferable embodiments, the controlled delivery dosing structure is adapted to controllably deliver the portion of the substance that comprises carbidopa contained by the controlled delivery dosing structure at a rate that is effective to, after a single administration of the dosage form to a patient, maintain a carbidopa plasma drug concentration that is at least about fifteen percent of the carbidopa Cmax throughout a window of at least about eighteen hours duration. In preferable embodiments, the controlled delivery dosing structure is adapted to controllably deliver the portion of the substance that comprises carbidopa contained by the controlled delivery dosing structure at a rate that is effective to, after a single administration of the dosage form to a patient, maintain a carbidopa plasma drug concentration that is at least about fifteen percent of the carbidopa Cmax throughout a window of at least about twenty hours duration.

Although the foregoing invention has been described in detail by way of example for purposes of clarity of understanding, it will be apparent to persons skilled in the art that certain changes and modifications are comprehended by the disclosure and can be practiced without undue experimentation within the scope of the appended claims, which are presented by way of illustration not limitation.

The inventive compositions are generally formulated as sterile, substantially isotonic and in full compliance with all Good Manufacturing Practice (GMP) regulations of the U.S. Food and Drug Administration.

All publications and patent documents cited above are hereby incorporated by reference in their entirety for all purposes to the same extent as if each were so individually denoted.

Each recited range includes all combinations and subcombinations of ranges, as well as specific numerals contained therein.

### EXAMPLES

The following examples are illustrative of the present invention and should not be considered as limiting the scope of the invention in any way, as these examples and other equivalents thereof will become apparent to those versed in the art in light of the present disclosure, drawings and accompanying claims.

### Example 1

### Preparation of Levodopa-Lauryl Sulfate Complex

1. The ion exchange column was packed with the cationic resin, DOWEX 50WX8-100 and a net weight of 117 g was obtained.

2. The column was rinsed with 70 mL deionized (DI) water (backflush), with care taken to not allow the column to dry out.

3. 5.768 g Sodium lauryl sulfate was dissolved in 577 mL DI water.

4. 175 mL DI water was passed through the column dropwise using a separatory funnel. Then the solution of step 3 was passed through the column dropwise using a separatory funnel and the eluate collected. After the SDS solution was passed through the column, a total of 70 mL DI water was used to rinse the column. The total sodium lauryl sulfate passed through was calculated to be less than the ion exchange resin's equilibrating point (capacity). The first eluate of 91 mL was discarded, the second eluate of 486 mL eluate was collected and used for complexation. The third 70 mL eluate was also discarded.

5. 3.323 g levodopa base was added to the second eluate, the mixture was stirred to solubilize levodopa at room temperature. The water was removed in vacuum oven at 40° C. After the sample was dried, 6.5 g product was collected. The product was in the form of a paste.

### Example 2

### Preparation of Levodopa-Tetradecyl Sulfate Complex

The following steps are carried out to form levodopa-tetradecyl sulfate complex. This complex is expected to have a higher melting point than the complex produced according to Example 1, and therefore may have greater utility in solid dosage forms.

The ion exchange column is packed with the cationic resin, Amberlyst 15 catalyst and a net weight of 19.16 g is targeted.

The column is rinsed with 20 mL deionized (DI) water (backflush), with care taken to not allow the column to dry out.

1.582 g Sodium tetradecyl sulfate is dissolved in 100 mL DI water.

The solution of step 3 is passed through the column dropwise using a separatory funnel and the eluate collected. After the sodium tetradecyl sulfate is passed through the column, a total of 20 mL DI water is used to rinse the column. The total sodium tetradecyl sulfate passed through is calculated to ensure that it is less than the ion exchange resin's equilibrating point (capacity).
a. Add 0.986 g levodopa to the eluate in step 4, stir the mixture to solubilize the levodopa at room temperature. Remove the water in vacuum oven at 40° C. After the sample was dried, a theoretical yield of 2.141 g product may be collected.

### Example 3

### Preparation of Carbidopa-Lauryl Sulfate Complex

1. The ion exchange column is packed with the cationic resin, DOWEX 50WX8-100 and a net weight of 117 g is targeted.

The column is rinsed with 70 mL deionized (DI) water (backflush), with care taken to not allow the column to dry out.

5.768 g Sodium lauryl sulfate is dissolved in 577 mL DI water.

Pass 175 mL DI water through the column dropwise using a separatory funnel. Then the solution of step 3 is passed through the column dropwise using a separatory funnel and the eluate collected. After the SDS solution is passed through the column, a total of 70 mL DI water is used to rinse the column. The total sodium lauryl sulfate passed through is calculated to ensure that it is less than the ion exchange resin's equilibrating point (capacity). The first eluate of 91 mL is discarded; the second eluate of 486 mL eluate is collected and used for complexation. The third 70 mL eluate is also discarded.

Add 4.116 g carbidopa monohydrate to the second eluate, stir the mixture to solubilize carbidopa at room temperature. Remove the water in vacuum oven at 40° C. After the sample is dried, a theoretical maximum of 8.3 g product may be obtained.

### Example 4

### Preparation of Carbidopa-Tetradecyl Sulfate Complex

The following steps are carried out to form carbidopa-tetradecyl sulfate complex. This complex is expected to have a higher melting point than the complex produced according to Example 3, and therefore may have greater utility in solid dosage forms.
1. The ion exchange column is packed with the cationic resin, DOWEX 50WX8-100 and a net weight of 117 g is targeted.
2. The column is rinsed with 70 mL deionized (DI) water (backflush), with care taken to not allow the column to dry out.
3. 5.768 g Sodium tetradecyl sulfate is dissolved in 577 mL DI water.
4. Pass 175 mL DI water through the column dropwise using a separatory funnel. Then the solution of step 3 is passed through the column dropwise using a separatory funnel and the eluate collected. After the Sodium tetradecyl solution is passed through the column, a total of 70 mL DI water is used to rinse the column. The total sodium tetradecyl sulfate passed through is calculated to ensure that it is less than the ion exchange resin's equilibrating point (capacity). The first eluate of 91 mL is discarded; the second eluate of 486 mL eluate is collected and used for complexation. The third 70 mL eluate is also discarded.
5. Add 3.752 g carbidopa monohydrate to the second eluate, stir the mixture to solubilize carbidopa at room temperature. Remove the water in vacuum oven at 40 ° C. After the sample is dried, a theoretical maximum of 7.94 g product may be obtained.

### Example 5

### Liquid Osmotic Dosage Form

A hard cap oral osmotic device system was manufactured for dispensing the complex of Example 1 in the G.I. tract. First, an osmotic push-layer formation was granulated with Glatt fluid bed granulator (FBG). The composition of the push granules was comprised of 63.67 wt % of polyethylene oxide of 7,000,000 molecular weight, 30.00 wt % sodium chloride, 1.00 wt % ferric oxide, 5.00 wt % hydroxypropylmethylcellulose of 9,200 molecular weight, 0.08 wt % butylated hydroxytoluene and 0.25 wt % magnesium stearate.

Second, the barrier layer granulation was produced using medium FBG. The composition of barrier-layer granules was comprised of 55 wt % Kollidon, 35 wt % Magnesium Stearate and 10 wt % EMM.

Third, the osmotic push layer granules and barrier layer granules were compressed into a bi-layer tablet with a Multi-layer Korsch press. 350 mg of the osmotic push-layer granules were added and tamped, then 100 mg of barrier layer granules were added onto and finally compressed under a force of 4500 N into a osmotic/barrier bi-layer tablet.

Fourth, 470 mg of the complex made according to Example 1 was dissolved into 108 mg propylene glycol (PG) using sonication at 45° C. for 5.5 h.

Next, Gelatin capsules (size 0) were subcoated with Surelease™. This will inhibit water-permeation into the capsulated liquid formulation during system operation. The subcoating is a membrane of ethylcellulose applied in the form of aqueous dispersion. The dispersion contained 25 wt % solids and was diluted to contain 15 wt % solids by adding purified water. The membrane weight of Surelease™ was 17 mg.

Next, a Surelease™ coated gelatin capsule was separated into two segments (body and cap). The drug-layer composition (578 mg) was filled into the capsule body.

Next, the osmotic/barrier tablet was placed in the filled capsule body.—Before inserting the engines into the capsules, a layer of sealing solution was applied around the barrier layer of the gelatin-coated bilayer engines. After engine insertion, a layer of banding solution was applied around the diameter at the interface of capsule and engine. This sealing and banding solution are the same, which is made of water/ethanol 50/50 wt %.

Next, the membrane composition comprising 80% cellulose acetate 398-10 and 20% Pluronic F-68 was dissolved in acetone with solid content of 5% in the coating solution. The solution was sprayed onto the pre-coating assemblies in a 12″ LDCS Hi-coater. After membrane coating, the systems were dried in oven at 45° C. for 24. The assemblies were coated with 131 mg of the rate-controlling membrane.

Next, a 30 mil (0.77 mm) exit orifice was drilled at the drug-layer side using a mechanical drill. Each system comprises 470 mg of the complex of Example 1. By adjusting the membrane weight, the release duration of the systems can be controlled.

### Example 6

Release Profile for Liquid Osmotic Dosage Form

The release rate for the dosage form made according to Example 5 was performed on the Distek 5100 (USP apparatus 2 paddle tester) in 900 mL artificial gastric fluid (AGF, pH=1.2). The temperature of the dissolution medium was maintained at 37° C. and the paddle speed was 100 rpm. The concentration of levodopa was measured with online UV spectroscopy at 280 nm. Two systems were tested.

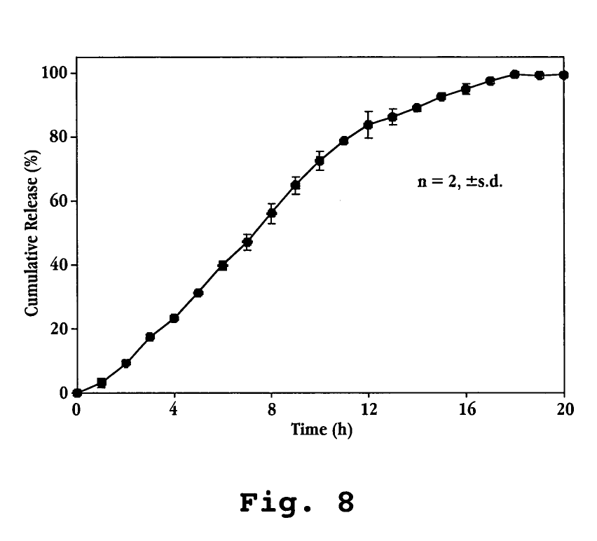

The results, showing cumulative release, are provided in FIG. 8.

### Example 7

Liquid Osmotic Dosage Form

A hard cap oral osmotic device system for dispensing the complexes of Example 1 and 3 in the G.I. tract may be prepared as follows:

First, an osmotic push-layer formation is granulated using a Glatt fluid bed granulator (FBG). The composition of the push granules is comprised of 63.67 wt % of polyethylene oxide of 7,000,000 molecular weight, 30.00 wt % sodium chloride, 1.00 wt % ferric oxide, 5.00 wt % hydroxypropylmethylcellulose of 9,200 molecular weight, 0.08 wt % butylated hydroxytoluene and 0.25 wt % magnesium stearate.

Second, the barrier layer granulations are produced using medium FBG. The composition of barrier-layer granules is comprised of 55 wt % Kollidon, 35 wt % Magnesium Stearate and 10 wt % EMM.

Third, the osmotic push layer granules and barrier layer granules are compressed into a bi-layer tablet with a Multi-layer Korsch press. 350 mg of the osmotic push-layer granules are added and tamped, then 100 mg of barrier layer granules are added onto and finally compressed under a force of 4500 N into a osmotic/barrier bi-layer tablet.

Fourth, 235 mg of the levodopa lauryl sulfate complex (100 mg levodopa equivalent) made according to Example 1, and 54 mg carbidopa lauryl sulfate complex (25 mg carbidopa equivalent) made according to Example 3 are dissolved into about 211 mg propylene glycol (PG) using sonication at 45° C. for ˜6 h.

Next, Gelatin capsules (size 0) are subcoated with Surelease™. This will inhibit water-permeation into the capsulated liquid formulation during system operation. The subcoating is a membrane of ethylcellulose applied in the form of aqueous dispersion. The dispersion contains 25 wt % solids and is diluted to contain 15 wt % solids by adding purified water. The membrane weight of Surelease™ is 17 mg.

Next, a Surelease(tm) coated gelatin capsule is separated into two segments (body and cap). The drug-layer composition (500 mg) is filled into the capsule body.

Next, the osmotic/barrier tablet is placed in the filled capsule body. Before inserting the engines into the capsules, a layer of sealing solution is applied around the barrier layer of the gelatin-coated bilayer engines. After engine insertion, a layer of banding solution is applied around the diameter at the interface of capsule and engine. This sealing and banding solution are the same, which is made of water/ethanol 50/50 wt %.

Next, the membrane composition comprising 80% cellulose acetate 398-10 and 20% Pluronic F-68 is dissolved in acetone with solid content of 5% in the coating solution. The solution is sprayed onto the pre-coating assemblies in a 12″ LDCS Hi-coater. After membrane coating, the systems are dried in oven at 45° C. for 24. The assemblies are coated with 131 mg of the rate-controlling membrane.

Next, a 30 mil (0.77 mm) exit orifice is drilled at the drug-layer side using a mechanical drill. By adjusting the membrane weight, the release duration of the systems can be controlled.

### Example 8

Preparation of Dosage Form Comprising a Levodopa-Tetradecyl Sulfate Complex and a Carbidopa-Tetradecyl Sulfate Complex

A dosage form is prepared as follows:

The levodopa-tetradecyl sulfate complex layer in the dosage form is prepared as follows. First, 7.56 grams of levodopa-tetradecyl sulfate complex, prepared as described in Example 2, 1.74 grams of carbidopa-tetradecyl sulfate complex, prepared as described in Example 4, 0.50 g polyethylene oxide of 5,000,000 molecular weight, 0.10 g of polyvinylpyrrolidone having molecular weight of about 38,000 are dry blended in a conventional blender for 20 minutes to yield a homogenous blend. Next, denatured anhydrous ethanol is added slowly to the blend with continuous mixing for 5 minutes. The blended wet composition is passed through a 16 mesh screen and dried overnight at room temperature. Then, the dry granules are passed through a 16 mesh screen and 0.10 g magnesium stearate are added and all the dry ingredients are dry blended for 5 minutes. The composition is comprised of 75.6 wt % levodopa-tetradecyl sulfate complex, 17.4 wt % carbidopa-tetradecyl sulfate complex, 5.0 wt % polyethylene oxide 5,000,000 molecular weight, 1.0 wt % polyvinylpyrrolidone having molecular weight of about 35,000 to 40,000 and 1.0 wt % magnesium stearate.

A push layer comprised of an osmopolymer hydrogel composition is prepared as follows. First, 637.70 g of pharmaceutically acceptable polyethylene oxide comprising a 7,000,000 molecular weight, 300 g sodium chloride and 10 g ferric oxide are separately screened through a 40 mesh screen. The screened ingredients are mixed with 50 g of hydroxypropylmethylcellulose of 9,200 molecular weight to produce a homogenous blend. Next, 150 mL of denatured anhydrous alcohol is added slowly to the blend with continuous mixing for 5 minutes. Then, 0.80 g of butylated hydroxytoluene is added followed by more blending. The freshly prepared granulation is passed through a 20 mesh screen and allowed to dry for 20 hours at room temperature (ambient). The dried ingredients are passed through a 20 mesh screen and 2.50 g of magnesium stearate is added and all the ingredients are blended for 5 minutes. The final composition is comprised of 63.67 wt % of polyethylene oxide, 30.00 wt % sodium chloride, 1.00 wt % ferric oxide, 5.00 wt % hydroxypropylmethylcellulose, 0.08 wt % butylated hydroxytoluene and 0.25 wt % magnesium stearate.

The bi-layer dosage form is prepared as follows. First, 654 mg of the drug layer composition is added to a punch and die set and tamped. Then, 327 mg of the hydrogel composition is added and the two layers compressed under a compression force of 1.0 ton (1000 kg) into a 9/32 inch (0.714 cm) diameter punch die set, forming an intimate bi-layered core (tablet).

A semipermeable wall-forming composition is prepared comprising 80.0 wt % cellulose acetate having a 39.8 % acetyl content and 20.0 % polyoxyethylene-polyoxypropylene copolymer having a molecular weight of 7680-9510 by dissolving the ingredients in acetone in a 80:20 wt/wt composition to make a 5.0% solids solution. Placing the solution container in a warm water bath during this step accelerates the dissolution of the components. The wall-forming composition is sprayed onto and around the bi-layered core to provide a 60 to 80 mg thickness semi-permeable wall.

Next, a 40 mil (1.02 mm) exit orifice is laser drilled in the semipermeable walled bi-layered tablet to provide contact of the drug containing layer with the exterior of the delivery device. The dosage form is dried to remove any residual solvent and water.

The release rate for the dosage form made according to Example 8 is performed on the Distek 5100 (USP apparatus 2 paddle tester) in 900 mL artificial gastric fluid (AGF, pH=1.2). The temperature of the dissolution medium was maintained at 37° C. and the paddle speed was 100 rpm. The concentration of levodopa was measured with online UV spectroscopy at 280 nm. Two systems are tested.

### Example 9

Modified Matrix Dosage Form

A matrix dosage form according to the present invention is prepared as follows. 247 grams of levodopa-tetradecyl sulfate complex, prepared as described in Example 2, 57 grams of carbidopa-tetradecyl sulfate complex, prepared as described in Example 4,—25 grams of hydroxypropyl methylcellulose having a number average molecular weight of 9,200 grams per mole, and 15 grams of hydroxypropyl methylcellulose having a molecular weight of 242,000 grams per mole, are passed through a screen having a mesh size of 40 wires per inch. The celluloses each have an average hydroxyl content of 8 weight percent and an average methoxyl content of 22 weight percent. The resulting sized powders are tumble mixed. Anhydrous ethyl alcohol is added slowly to the mixed powders with stirring until a dough consistency is produced. The damp mass is then extruded through a 20 mesh screen and air dried overnight. The resulting dried material is re-screened through a 20 mesh screen to form the final granules. 2 grams of the tableting lubricant, magnesium stearate, which are sized through an 80 mesh screen, are then tumbled into the granules. 663 mg of the resulting granulation is placed in a die cavity having an inside diameter of 9/32 inch and compressed with deep concave punch tooling using a pressure head of 2 tons. This forms a longitudinal capsule core having an overall length, including the rounded ends, of 0.691 inch. The cylindrical body of the capsule, from tablet land to tablet land, span a distance of 12 mm. Each core contains a unit dose of levodopa-tetradecyl sulfate complex of 495 mg (200 mg levodopa equivalent) and carbidopa tetradecyl sulfate complex of 114 mg (50 mg carbidopa equivalent).

Next, rings of polyethylene having an inside diameter of 9/32 inch, a wall thickness of 0.013 inch, and a width of 2 mm are then fabricated. These rings, or bands, are press fitted onto the core to complete the dosage form.

### Example 10

In Vivo Colonic Absorption Using Flushed Ligated Colonic Model in Rats

An animal model commonly known as the “intracolonic ligated model” was employed for testing formulations. Surgical preparation of a fasted anesthetized 0.3-0.5 kg Sprague-Dawley male rats proceeded as follows. A segment of proximal colon was isolated and the colon was flushed of fecal materials. The segment was ligated at both ends while a catheter was placed in the lumen and exteriorized above the skin for delivery of test formulation. The colonic contents were flushed out and the colon was returned to the abdomen of the animal. Depending on the experimental set up, the test formulation was added after the segment was filled with 1 mL/kg of 20 mM sodium phosphate buffer, pH 7.4, to more accurately simulate the actual colon environment in a clinical situation.

Rats were allowed to equilibrate for approximately 1 hour after surgical preparation and prior to exposure to each test formulation. The test compounds were administered as an intracolonic bolus and delivered at 2 mg levodopa/rat or 2 mg levodopa lauryl sulfate/rat. Blood samples were obtained from the jugular catheter at 0, 15, 30, 60, 90, 120, 180 and 240 minutes after administration of the test formulation and analyzed for blood levodopa concentration.

Another group of rats were treated with levodopa intravenously, at a dose of 0.4 mg/kg. Blood samples were withdrawn at the same times indicated above for analysis of levodopa concentration.

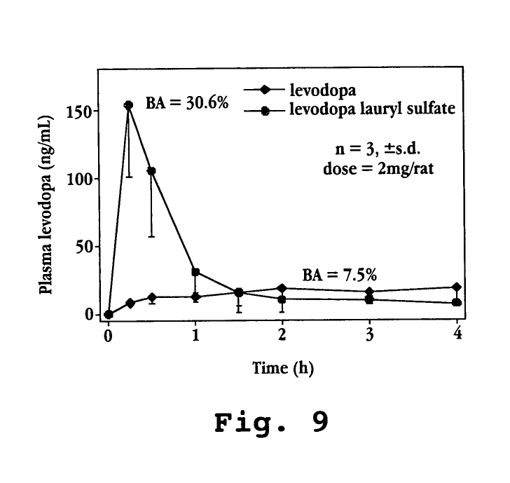

The levodopa plasma concentration for each test animal, and the average plasma concentration for animals in each test group, are shown in Tables A-C. FIG. 9 shows the average levodopa concentration in each test group as a function of time.

TABLE A
Levodopa colonic: 2 mg levodopa/rat, Plasma level (ng/ml)

| time (h) | Rat1 | Rat2 | Rat3 |
|---|---|---|---|
| 0 | 0 | 0 | 0 |
| 0.25 | 8.9 | 9.42 | 6.09 |
| 0.5 | 10.8 | 17.7 | 9.18 |
| 1 | 8.59 | 12.8 | 17.2 |
| 1.5 | 10.4 | 9.32 | 26.5 |
| 2 | 17.6 | 12.9 | 24.9 |
| 3 | 18.8 | 13 | 14.5 |
| 4 | 32.5 | 13.1 | 9.61 |

TABLE B
Levodopa lauryl sulfate colonic: 2 mg levodopa lauryl sulfate/rat (0.85 mg
levodopa equivalent/rat), Plasma level (ng/ml)

| time (h) | Rat1 | Rat2 | Rat3 |
|---|---|---|---|
| 0 | 0 | 0 | 0 |
| 0.25 | 92.3 | 182 | 187 |
| 0.5 | 48.8 | 137 | 130 |
| 1 | 15.9 | 29.3 | 47.1 |
| 1.5 | 0 | 17.8 | 28.6 |
| 2 | 0 | 11 | 19.1 |
| 3 | 8.15 | 13.6 | 7.2 |
| 4 | 5.82 | 7.49 | 7.28 |

TABLE C
Levodopa iv: 0.4 mg levodopa/kg, Plasma level (ng/ml)

| time (h) | Rat1 | Rat2 | Rat3 |
|---|---|---|---|
| 0 | 0 | 0 | 0 |
| 0.033 | 336 | 247 | 690 |
| 0.167 | 53.2 | 53.6 | 110 |
| 0.5 | 16 | 11.4 | 41.6 |
| 1 | 7.34 | 11.5 | 18.2 |
| 1.5 | 6.51 | 0 | 13.8 |
| 2 | 0 | 5.82 | 8.23 |
| 3 | 0 | 0 | 5.53 |

### Example 11

In Vivo Duodenal Absorption

Nine rats were randomized into three test groups (n=3). Levodopa or levodopa-lauryl sulfate complex, prepared as described in Example 1, in a saline vehicle were intubated into the beginning of the duodenum of rats at dosages of at 2 mg levodopa/rat or 2 mg levodopa lauryl sulfate/rat. The remaining test group was given 0.4 mg/kg levodopa intravenously.

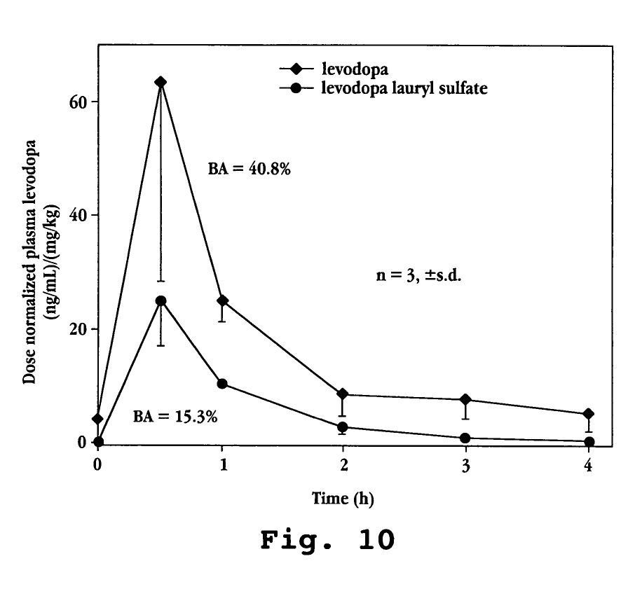

Blood samples were taken from each animal over a three or four hour period and analyzed for levodopa content. The results are shown in Tables D-F and in FIG. 10.

TABLE D
Levodopa iv: 0.4 mg levodopa/kg, Plasma level (ng/ml)

| time (h) | Rat1 | Rat2 | Rat3 |
|---|---|---|---|
| 0 | 0 | 0 | 0 |
| 0.033 | 336 | 247 | 690 |
| 0.167 | 53.2 | 53.6 | 110 |
| 0.5 | 16 | 11.4 | 41.6 |
| 1 | 7.34 | 11.5 | 18.2 |
| 1.5 | 6.51 | 0 | 13.8 |
| 2 | 0 | 5.82 | 8.23 |
| 3 | 0 | 0 | 5.53 |

TABLE E
Levodopa duodenal: 2 mg levodopa/rat, Plasma level (ng/ml)

| time (h) | rat1 | rat2 | rat3 |
|---|---|---|---|
| 0 | 0 | 0 | 7.57 |
| 0.5 | 159 | 143 | 80.2 |
| 1 | 48.6 | 52.9 | 56.5 |
| 2 | 9.48 | 22.3 | 13 |
| 3 | 5.73 | 6.26 | 6.28 |
| 4 | 0 | 5.06 | 5.97 |

TABLE F
Levodopa lauryl sulfate duodenal: 2 mg levodopa lauryl sulfate/rat
(0.85 mg levodopa equivalent/rat), Plasma level (ng/ml)

| | | | |
|---|---|---|---|
| 0 | 9.42 | 8.7 | 8.55 |
| 0.5 | 210 | 113 | 65.6 |
| 1 | 57.3 | 51.3 | 43.7 |
| 2 | 21.3 | 22 | 9.24 |
| 3 | 12 | 11.6 | 24 |
| 4 | 8.07 | 7.45 | 19.6 |

1. A substance comprising:
a complex comprising levodopa and a transport moiety.

2. The substance of claim 1, wherein the transport moiety comprises an alkyl sulfate salt.

3. The substance of claim 2, wherein the alkyl sulfate salt comprises sodium lauryl sulfate.

4. A pharmaceutical composition comprising:
the substance of claim 1 and a pharmaceutically-acceptable carrier.

5. An oral dosage form comprising the pharmaceutical composition of claim 4.

6. The oral dosage form of claim 5, wherein the oral dosage form further comprises carbidopa.

7. The oral dosage form of claim 5, wherein the oral dosage form further comprises a carbidopa complex.

8. The oral dosage form of claim 5, wherein the oral dosage form comprises an oral controlled delivery dosage form.

9. The oral dosage form of claim 8, wherein the oral dosage form comprises an osmotic oral controlled delivery dosage form.

10. The oral dosage form of claim 9, wherein the osmotic oral controlled delivery dosage form comprises a solid osmotic oral controlled delivery dosage form.

11. The oral dosage form of claim 10, wherein the solid osmotic oral controlled delivery dosage form further comprises carbidopa.

12. The oral dosage form of claim 10, wherein the solid osmotic oral controlled delivery dosage form further comprises a carbidopa complex.

13. The oral dosage form of claim 9, wherein the osmotic oral controlled delivery dosage form comprises a liquid osmotic oral controlled delivery dosage form.

14. The oral dosage form of claim 13, wherein the liquid osmotic oral controlled delivery dosage form further comprises carbidopa.

15. The oral dosage form of claim 13, wherein the liquid osmotic oral controlled delivery dosage form further comprises a carbidopa complex.

16. A method comprising:
administering the oral dosage form of claim 5 to a patient.

17. A method comprising:
administering the oral dosage form of claim 6 to a patient.

18. A method comprising:
administering the oral dosage form of claim 7 to a patient.

19. A method comprising:
administering the oral dosage form of claim 8 to a patient.

20. A method comprising:
administering the oral dosage form of claim 9 to a patient.

21. A method comprising:
administering the oral dosage form of claim 10 to a patient.

22. A method comprising:
administering the oral dosage form of claim 11 to a patient.

23. A method comprising:
administering the oral dosage form of claim 12 to a patient.

24. A method comprising:
administering the oral dosage form of claim 13 to a patient.

25. A method comprising:
administering the oral dosage form of claim 14 to a patient.

26. A method comprising:
administering the oral dosage form of claim 15 to a patient.

27. The oral dosage form of claim 8, wherein the controlled delivery dosage form controllably delivers the substance in a delivery dose pattern of from about 0 wt % to about 20 wt % in about 0 to about 4 hrs, about 20 wt % to about 50 wt % in about 0 to about 8 hrs, about 55 wt % to about 85 wt % in about 0 to about 14 hrs, and about 80 wt % to about 100 wt % in about 0 to about 24 hrs.

28. The oral dosage form of claim 8, wherein the controlled delivery dosage form controllably delivers the substance in a delivery dose pattern of from about 0 wt % to about 20 wt % in about 0 to about 4 hrs, about 20 wt % to about 50 wt % in about 0 to about 8 hrs, about 55 wt % to about 85 wt % in about 0 to about 14 hrs, and about 80 wt % to about 100 wt % in about 0 to about 20 hrs.

29. The oral dosage form of claim 8, wherein the controlled delivery dosage form controllably delivers the substance in a delivery dose pattern of from about 0 wt % to about 20 wt % in about 0 to about 2 hrs, about 20 wt % to about 50 wt % in about 0 to about 4 hrs, about 55 wt % to about 85 wt % in about 0 to about 7 hrs, and about 80 wt % to about 100 wt % in about 0 to about 8 hrs.

30. A method comprising:
providing an alkyl sulfate salt;
converting the alkyl sulfate salt to an acid form of the alkyl sulfate;
contacting levodopa with the acid form of the alkyl sulfate to form a levodopa-alkyl sulfate complex; and
isolating the complex.

31. The method of claim 30, wherein the alkyl sulfate salt comprises sodium lauryl sulfate.

32. The method of claim 30, wherein converting the alkyl sulfate salt to an acid form of the alkyl sulfate is performed using an ion exchange process.

33. A substance comprising:
a complex comprising carbidopa and a transport moiety.

34. The substance of claim 33, wherein the transport moiety comprises an alkyl sulfate salt.

35. The substance of claim 34, wherein the alkyl sulfate salt comprises sodium lauryl sulfate.

36. A pharmaceutical composition comprising:
the substance of claim 33 and a pharmaceutically-acceptable carrier.

37. An oral dosage form comprising the pharmaceutical composition of claim 36.

38. The oral dosage form of claim 37, wherein the oral dosage form further comprises levodopa.

39. The oral dosage form of claim 37, wherein the oral dosage form further comprises a levodopa complex.

40. The oral dosage form of claim 37, wherein the oral dosage form comprises a controlled delivery oral dosage form.

41. The oral dosage form of claim 40, wherein the oral dosage form comprises an osmotic controlled delivery oral dosage form.

42. The oral dosage form of claim 41, wherein the osmotic controlled delivery oral dosage form comprises a solid osmotic controlled delivery oral dosage form.

43. The oral dosage form of claim 42, wherein the solid osmotic controlled delivery oral dosage form further comprises levodopa.

44. The oral dosage form of claim 42, wherein the solid osmotic controlled delivery oral dosage form further comprises a levodopa complex.

45. The oral dosage form of claim 39, wherein the osmotic controlled delivery oral dosage form comprises a liquid osmotic controlled delivery oral dosage form.

46. The oral dosage form of claim 45, wherein the liquid osmotic controlled delivery oral dosage form further comprises levodopa.

47. The oral dosage form of claim 45, wherein the liquid osmotic controlled delivery oral dosage form further comprises a levodopa complex.

48. A method comprising:
administering the oral dosage form of claim 37 to a patient.

49. A method comprising:
administering the oral dosage form of claim 38 to a patient.

50. A method comprising:
administering the oral dosage form of claim 39 to a patient.

51. A method comprising:
administering the oral dosage form of claim 40 to a patient.

52. A method comprising:
administering the oral dosage form of claim 41 to a patient.

53. A method comprising:
administering the oral dosage form of claim 42 to a patient.

54. A method comprising:
administering the oral dosage form of claim 43 to a patient.

55. A method comprising:
administering the oral dosage form of claim 44 to a patient.

56. A method comprising:
administering the oral dosage form of claim 45 to a patient.

57. A method comprising:
administering the oral dosage form of claim 46 to a patient.

58 A method comprising:
administering the oral dosage form of claim 47 to a patient.

59. The oral dosage form of claim 40, wherein the controlled delivery dosage form controllably delivers the substance in a delivery dose pattern of from about 0 wt % to about 20 wt % in about 0 to about 4 hrs, about 20 wt % to about 50 wt % in about 0 to about 8 hrs, about 55 wt % to about 85 wt % in about 0 to about 14 hrs, and about 80 wt % to about 100 wt % in about 0 to about 24 hrs.

60. The oral dosage form of claim 40, wherein the controlled delivery dosage form controllably delivers the substance in a delivery dose pattern of from about 0 wt % to about 20 wt % in about 0 to about 4 hrs, about 20 wt % to about 50 wt % in about 0 to about 8 hrs, about 55 wt % to about 85 wt % in about 0 to about 14 hrs, and about 80 wt % to about 100 wt % in about 0 to about 20 hrs.

61. The oral dosage form of claim 40, wherein the controlled delivery dosage form controllably delivers the substance in a delivery dose pattern of from about 0 wt % to about 20 wt % in about 0 to about 2 hrs, about 20 wt % to about 50 wt % in about 0 to about 4 hrs, about 55 wt % to about 85 wt % in about 0 to about 7 hrs, and about 80 wt % to about 100 wt % in about 0 to about 8 hrs.

62. A method comprising:
providing an alkyl sulfate salt;
converting the alkyl sulfate salt to an acid form of the alkyl sulfate;
contacting carbidopa with the acid form of the alkyl sulfate to form a levodopa-alkyl sulfate complex; and
isolating the complex.

63. The method of claim 62, wherein the alkyl sulfate salt comprises sodium lauryl sulfate.

64. The method of claim 63, wherein converting the alkyl sulfate salt to an acid form of the alkyl sulfate is performed using an ion exchange process.

65. An oral dosage form comprising:
(i) an oral controlled delivery dosing structure comprising structure that controllably delivers a substance that comprises levodopa and a substance that comprises carbidopa; wherein at least a portion of the substance that comprises levodopa and a portion of the substance that comprises carbidopa are contained by the controlled delivery dosing structure; and
wherein the controlled delivery dosing structure is adapted to controllably deliver the portion of the substance that comprises levodopa and the portion of the substance that comprises carbidopa contained by the controlled delivery dosing structure at rates that are effective to, after a single administration of the dosage form to a patient:
a. provide a levodopa Cmax ranging from about 236 to about 988 ng/mL,
b. provide a levodopa AUC from about 3676 to about 15808 h·ng/mL, and
c. maintain a levodopa plasma drug concentration that is at least about fifteen percent of the levodopa Cmax throughout a window of at least about ten hours duration.
d. provide a carbidopa Cmax ranging from about 1 to about 500 ng/ml μmol/L,
e. provide an carbidopa AUC from about 20000 to about 200000 h·ng/mL, and
f. maintain a carbidopa plasma drug concentration that is at least about fifteen percent of the carbidopa Cmax throughout a window of at least about ten hours duration.

66. The oral dosage form of claim 65, wherein the substance that comprises levodopa comprises:
a levodopa complex.

67. The oral dosage form of claim 65, wherein the substance that comprises levodopa comprises:
a levodopa prodrug.

68. The oral dosage form of claim 65, wherein the substance that comprises carbidopa comprises:
a carbidopa complex.

69. The oral dosage form of claim 65, wherein the substance that comprises carbidopa comprises:
a carbidopa prodrug.

70. The oral dosage form of claim 65, wherein the controlled delivery dosing structure is adapted to controllably deliver the portion of the substance that comprises levodopa contained by the controlled delivery dosing structure at a rate that is effective to, after a single administration of the dosage form to a patient, maintain a levodopa plasma drug concentration that is at least about fifteen percent of the Cmax throughout a window of at least about twelve hours duration.

71. The oral dosage form of claim 70, wherein the controlled delivery dosing structure is adapted to controllably deliver the portion of the substance that comprises levodopa contained by the controlled delivery dosing structure at a rate that is effective to, after a single administration of the dosage form to a patient, maintain a levodopa plasma drug concentration that is at least about fifteen percent of the Cmax throughout a window of at least about sixteen hours duration.

72. The oral dosage form of claim 71, wherein the controlled delivery dosing structure is adapted to controllably deliver the portion of the substance that comprises levodopa contained by the controlled delivery dosing structure at a rate that is effective to, after a single administration of the dosage form to a patient, maintain a levodopa plasma drug concentration that is at least about fifteen percent of the Cmax throughout a window of at least about eighteen hours duration.

73. The oral dosage form of claim 72, wherein the controlled delivery dosing structure is adapted to controllably deliver the portion of the substance that comprises levodopa contained by the controlled delivery dosing structure at a rate that is effective to, after a single administration of the dosage form to a patient, maintain a levodopa plasma drug concentration that is at least about fifteen percent of the Cmax throughout a window of at least about twenty hours duration.

74. The oral dosage form of claim 65, wherein the controlled delivery dosing structure is adapted to controllably deliver the portion of the substance that comprises carbidopa contained by the controlled delivery dosing structure at a rate that is effective to, after a single administration of the dosage form to a patient, maintain a carbidopa plasma drug concentration that is at least about fifteen percent of the Cmax throughout a window of at least about twelve hours duration.

75. The oral dosage form of claim 74, wherein the controlled delivery dosing structure is adapted to controllably deliver the portion of the substance that comprises carbidopa contained by the controlled delivery dosing structure at a rate that is effective to, after a single administration of the dosage form to a patient, maintain a carbidopa plasma drug concentration that is at least about fifteen percent of the Cmax throughout a window of at least about sixteen hours duration.

76. The oral dosage form of claim 75, wherein the controlled delivery dosing structure is adapted to controllably deliver the portion of the substance that comprises carbidopa contained by the controlled delivery dosing structure at a rate that is effective to, after a single administration of the dosage form to a patient, maintain a carbidopa plasma drug concentration that is at least about fifteen percent of the Cmax throughout a window of at least about eighteen hours duration.

77. The oral dosage form of claim 76, wherein the controlled delivery dosing structure is adapted to controllably deliver the portion of the substance that comprises carbidopa contained by the controlled delivery dosing structure at a rate that is effective to, after a single administration of the dosage form to a patient, maintain a carbidopa plasma drug concentration that is at least about fifteen percent of the Cmax throughout a window of at least about twenty hours duration.

78. An oral controlled delivery dosage form comprising
an oral controlled delivery dosing structure comprising structure that controllably delivers a substance that comprises levodopa;
wherein at least a portion of the substance that comprises levodopa is contained by the controlled delivery dosing structure; and
wherein the controlled delivery dosing structure is adapted to controllably deliver the portion of the substance that comprises levodopa contained by the controlled delivery dosing structure at an ascending rate of release effective to, after a single administration of the dosage form to a patient, provide a substantially zero order levodopa plasma profile for a window of at least about six hours duration.

79. The oral controlled delivery dosage form of claim 78, wherein the controlled delivery dosing structure is adapted to controllably deliver the portion of the substance that comprises levodopa contained by the controlled delivery dosing structure at an ascending rate of release effective to, after a single administration of the dosage form to a patient, provide a substantially zero order levodopa plasma profile for a window of at least about twelve hours duration.

80. The oral controlled delivery dosage form of claim 79, wherein the controlled delivery dosing structure is adapted to controllably deliver the portion of the substance that comprises levodopa contained by the controlled delivery dosing structure at an ascending rate of release effective to, after a single administration of the dosage form to a patient, provide a substantially zero order levodopa plasma profile for a window of at least about sixteen hours duration.

81. The oral dosage form of claim 78, wherein the substance that comprises levodopa comprises:
a levodopa complex.

82. The oral dosage form of claim 78, wherein the substance that comprises levodopa comprises:
a levodopa prodrug.

83. The oral controlled delivery dosage form of claim 78 further comprising:
an oral controlled delivery dosing structure comprising structure that controllably delivers a substance that comprises carbidopa;
wherein at least a portion of the substance that comprises carbidopa is contained by the controlled delivery dosing structure; and
wherein the controlled delivery dosing structure is adapted to controllably deliver the portion of the substance that comprises carbidopa contained by the controlled delivery dosing structure at an ascending rate of release effective to, after a single administration of the dosage form to a patient, provide a substantially zero order carbidopa plasma profile for a window of at least about six hours duration.

84. The oral controlled delivery dosage form of claim 83, wherein the controlled delivery dosing structure is adapted to controllably deliver the portion of the substance that comprises carbidopa contained by the controlled delivery dosing structure at an ascending rate of release effective to, after a single administration of the dosage form to a patient, provide a substantially zero order carbidopa plasma profile for a window of at least about twelve hours duration.

85. The oral controlled delivery dosage form of claim 84, wherein the controlled delivery dosing structure is adapted to controllably deliver the portion of the substance that comprises carbidopa contained by the controlled delivery dosing structure at an ascending rate of release effective to, after a single administration of the dosage form to a patient, provide a substantially zero order carbidopa plasma profile for a window of at least about sixteen hours duration.

86. The oral dosage form of claim 83, wherein the substance that comprises carbidopa comprises:
a carbidopa complex.

87. The oral dosage form of claim 83, wherein the substance that comprises carbidopa comprises:
a carbidopa prodrug.

88. A composition comprising:
levodopa;
an alkyl sulfate salt; and
a pharmaceutically-acceptable carrier.

89. The composition of claim 88, wherein the alkyl sulfate salt comprises sodium lauryl sulfate.

90. An oral dosage form comprising the pharmaceutical composition of claim 88.

91. The oral dosage form of claim 90, wherein the oral dosage form further comprises carbidopa.

92. An oral dosage form comprising:
(i) an oral controlled delivery dosing structure comprising structure that controllably delivers a substance that comprises levodopa;
wherein at least a portion of the substance that comprises levodopa is contained by the controlled delivery dosing structure; and
wherein the controlled delivery dosing structure is adapted to controllably deliver the portion of the substance that comprises levodopa contained by the controlled delivery dosing structure at rates that are effective to, after a single administration of the dosage form to a patient:
a. provide a levodopa Cmax ranging from about 236 to about 988 ng/mL,
b. provide a levodopa AUC from about 3676 to about 15808 h·ng/mL, and
c. maintain a levodopa plasma drug concentration that is at least about fifteen percent of the levodopa Cmax throughout a window of at least about ten hours duration.

93. The oral dosage form of claim 92, wherein the substance that comprises levodopa comprises:
a levodopa complex.

94. The oral dosage form of claim 92, wherein the substance that comprises levodopa comprises:
a levodopa prodrug.

95. The oral dosage form of claim 92, wherein the controlled delivery dosing structure is adapted to controllably deliver the portion of the substance that comprises levodopa contained by the controlled delivery dosing structure at a rate that is effective to, after a single administration of the dosage form to a patient, maintain a levodopa plasma drug concentration that is at least about fifteen percent of the levodopa Cmax throughout a window of at least about twelve hours duration.

96. The oral dosage form of claim 92, wherein the controlled delivery dosing structure is adapted to controllably deliver the portion of the substance that comprises levodopa contained by the controlled delivery dosing structure at a rate that is effective to, after a single administration of the dosage form to a patient, maintain a levodopa plasma drug concentration that is at least about fifteen percent of the levodopa Cmax throughout a window of at least about sixteen hours duration.

97. The oral dosage form of claim 92, wherein the controlled delivery dosing structure is adapted to controllably deliver the portion of the substance that comprises levodopa contained by the controlled delivery dosing structure at a rate that is effective to, after a single administration of the dosage form to a patient, maintain a levodopa plasma drug concentration that is at least about fifteen percent of the levodopa Cmax throughout a window of at least about eighteen hours duration.

98. The oral dosage form of claim 92, wherein the controlled delivery dosing structure is adapted to controllably deliver the portion of the substance that comprises levodopa contained by the controlled delivery dosing structure at a rate that is effective to, after a single administration of the dosage form to a patient, maintain a levodopa plasma drug concentration that is at least about fifteen percent of the levodopa Cmax throughout a window of at least about twenty hours duration.

99. An oral dosage form comprising:
(i) an oral controlled delivery dosing structure comprising structure that controllably delivers a substance that comprises carbidopa;
wherein at least a portion of the substance that comprises carbidopa is contained by the controlled delivery dosing structure; and
wherein the controlled delivery dosing structure is adapted to controllably deliver the portion of the substance that comprises carbidopa contained by the controlled delivery dosing structure at rates that are effective to, after a single administration of the dosage form to a patient:
a. provide a carbidopa Cmax ranging from about 1 to about 500 ng/ml μmol/L,
b. provide an carbidopa AUC from about 20000 to about 200000 h·ng/mL, and
c. maintain a carbidopa plasma drug concentration that is at least about fifteen percent of the carbidopa Cmax throughout a window of at least about ten hours duration.

100. The oral dosage form of claim 99, wherein the substance that comprises carbidopa comprises:
a carbidopa complex.

101. The oral dosage form of claim 99, wherein the substance that comprises carbidopa comprises:
a carbidopa prodrug.

102. The oral dosage form of claim 99, wherein the controlled delivery dosing structure is adapted to controllably deliver the portion of the substance that comprises carbidopa contained by the controlled delivery dosing structure at a rate that is effective to, after a single administration of the dosage form to a patient, maintain a carbidopa plasma drug concentration that is at least about fifteen percent of the carbidopa Cmax throughout a window of at least about twelve hours duration.

103. The oral dosage form of claim 99, wherein the controlled delivery dosing structure is adapted to controllably deliver the portion of the substance that comprises carbidopa contained by the controlled delivery dosing structure at a rate that is effective to, after a single administration of the dosage form to a patient, maintain a carbidopa plasma drug concentration that is at least about fifteen percent of the carbidopa Cmax throughout a window of at least about sixteen hours duration.

104. The oral dosage form of claim 99, wherein the controlled delivery dosing structure is adapted to controllably deliver the portion of the substance that comprises carbidopa contained by the controlled delivery dosing structure at a rate that is effective to, after a single administration of the dosage form to a patient, maintain a carbidopa plasma drug concentration that is at least about fifteen percent of the carbidopa Cmax throughout a window of at least about eighteen hours duration.

105. The oral dosage form of claim 99, wherein the controlled delivery dosing structure is adapted to controllably deliver the portion of the substance that comprises carbidopa contained by the controlled delivery dosing structure at a rate that is effective to, after a single administration of the dosage form to a patient, maintain a carbidopa plasma drug concentration that is at least about fifteen percent of the carbidopa Cmax throughout a window of at least about twenty hours duration.
